(19) United States
(12) Patent Application Publication
(10) Pub. No.: US 2023/0200785 A1
(43) Pub. Date: Jun. 29, 2023

(54) ULTRASOUND DIAGNOSIS APPARATUS, IMAGE PROCESSING APPARATUS, AND IMAGE PROCESSING METHOD

(71) Applicant: CANON MEDICAL SYSTEMS CORPORATION, Tochigi (JP)

(72) Inventors: Yasuhiko ABE, Otawara (JP); Shinichi HASHIMOTO, Otawara (JP); Kazuya AKAKI, Utsunomiya (JP)

(73) Assignee: CANON MEDICAL SYSTEMS CORPORATION, Tochigi (JP)

(21) Appl. No.: 18/179,156

(22) Filed: Mar. 6, 2023

Related U.S. Application Data
(63) Continuation of application No. 14/498,249, filed on Sep. 26, 2014, now abandoned.
Application No. 14/498,249 is a continuation of application No. PCT/JP2013/058641, filed on Mar. 25, 2013.

(30) Foreign Application Priority Data
Mar. 30, 2012 (JP) 2012-082164
Mar. 25, 2013 (JP) 2013-062787

(51) Int. Cl. A61B 8/08; A61B 8/00; A61B 8/06; G06T 7/246; G06T 7/62; A61B 8/14

(52) U.S. Cl. CPC A61B 8/5223; A61B 8/0883; A61B 8/467; A61B 8/543; A61B 8/0858; A61B 8/463; A61B 5/1075; G06T 7/251; G06T 7/62; A61B 8/14; G06T 2207/30048; A61B 8/488; G06T 2207/10132; A61B 8/06

(57) ABSTRACT

An ultrasonic diagnostic apparatus according to an embodiment includes an image obtaining unit, a contour position obtaining unit, a volume information calculating unit, and a controlling unit. The image obtaining unit obtains a plurality of groups of two-dimensional ultrasound image data each of which is generated by performing ultrasound scans, the ultrasound scans being performed on each of a plurality of predetermined cross-sectional planes, and performed for predetermined time. The contour position obtaining unit obtains, by performing a tracking process over the predetermined time period, time-series data of contour positions, the contour positions being either one of, or both of, a cavity interior and a cavity exterior of a predetermined site. The volume information calculating unit calculates, on a basis of a plurality of the time-series data of contour positions, volume information of the predetermined site. The controlling unit exercises control so as to output the volume information.

### CROSS-REFERENCE TO RELATED APPLICATIONS

This application is a continuation of U.S. application Ser. No. 14/498,249, filed Sep. 26, 2014, which is a continuation of PCT international application Ser. No. PCT/JP2013/058641 filed on Mar. 25, 2013 which designates the United States, incorporated herein by reference, and which claims the benefit of priority from Japanese Patent Application No. 2012-082164, filed on Mar. 30, 2012; and Japanese Patent Application No. 2013-062787, filed on Mar. 25, 2013, the entire contents of all of which are incorporated herein by reference.

### FIELD

Embodiments described herein relate generally to an ultrasound diagnosis apparatus, an image processing apparatus, and an image processing method.

### BACKGROUND

Volume information of the heart is an important determinant factor for a prognosis of heart failure and is known to be information that is essential to selecting a treatment plan. Examples of volume information of the heart include a volume of the left ventricular cavity interior, a volume of the left atrial cavity interior, and a myocardial mass of the left ventricle. During an echocardiography, measuring of these types of volume information is mainly performed by implementing an M-mode method.

The volume measuring process using the M-mode method is commonly used in the actual clinical field, because the process is simple where a distance is measured in two time phases within M-mode images corresponding to one or more heartbeats. The M-mode images are acquired by using a parasternal long-axis (P-LAX) approach by which, for example, a long-axis cross-sectional plane is scanned. According to the M-mode method, however, because the volume is estimated on the basis of one-dimensional M-mode images, there are some situations where the estimated information contains a large error. In those situations, there is a possibility that an erroneous detection may occur where a group that requires no treatment is detected as a group that requires a treatment. In addition, there is a possibility that a group that requires a treatment may be overlooked.

To cope with these situations, when volume information is measured by using a “modified-Simpson's method”, the level of precision is known to be sufficiently high in practice, even with medical cases exhibiting a regional wall motion abnormality (e.g., medical cases where the shape of the cavity interior is complicated). The “modified-Simpson's method” is a method by which a volume is estimated by using contour information of myocardia rendered in two-dimensional image data taken on each of two mutually-different cross-sectional planes. The “modified-Simpson's method” is known to be able to achieve a precision level that is approximately equal to that of a “cardiac Magnetic Resonance Imaging (MRI)” process.

For example, when a volume is estimated by using the “modified-Simpson's method”, ultrasound image data (two-dimensional B-mode image data) taken on two cross-sectional planes such as an apical four-chamber view (hereinafter, “A4C view”) and an apical two-chamber view (hereinafter, “A2C view”) is used. However, the “modified-Simpson's method” is not widely used in the actual clinical field, because processes that are manually performed by an operator are cumbersome and require a lot of labor from the operator.

### BRIEF DESCRIPTION OF THE DRAWINGS

FIG. 1 is a block diagram of an exemplary configuration of an ultrasound diagnosis apparatus according to a first embodiment;

FIG. 2 is a drawing for explaining a disc summation method (a Simpson's method);

FIG. 3 is a drawing for explaining a modified-Simpson's method;

FIG. 4 is a block diagram of an exemplary configuration of an image processing unit according to the first embodiment;

FIG. 5 is a drawing for explaining an image obtaining unit according to the first embodiment;

FIG. 6 is a drawing for explaining an example of a two-dimensional speckle tracking process;

FIG. 7 is a table of examples of volume information calculated by a volume information calculating unit according to the first embodiment;

FIG. 8 is a chart for explaining a detecting unit according to the first embodiment;

FIG. 9 is a flowchart for explaining an example of a process performed by the ultrasound diagnosis apparatus according to the first embodiment;

FIG. 10 is a drawing for explaining a first modification example of the first embodiment;

FIG. 11A and FIG. 11B are drawings for explaining a second modification example of the first embodiment;

FIG. 12 is a drawing for explaining a detecting unit according to a second embodiment;

FIG. 13 is a flowchart for explaining an example of a volume information calculating process performed by an ultrasound diagnosis apparatus according to the second embodiment;

FIG. 14 is a flowchart for explaining an example of a volume information re-calculating process performed by the ultrasound diagnosis apparatus according to the second embodiment;

FIG. 15 is a drawing for explaining a modification example of the second embodiment;

FIG. 16 and FIG. 17 are drawings for explaining a contour position obtaining unit according to a third embodiment;

FIG. 18 is a flowchart for explaining an example of a process performed by an ultrasound diagnosis apparatus according to the third embodiment;

FIG. 19 is a block diagram of an exemplary configuration of an image processing unit according to a fourth embodiment;

FIG. 20 is a drawing of an example of information that is output according to the fourth embodiment; and

FIG. 21 is a flowchart for explaining an example of a process performed by an ultrasound diagnosis apparatus according to the fourth embodiment.

### DETAILED DESCRIPTION

An ultrasonic diagnostic apparatus according to an embodiment includes an image obtaining unit, a contour position obtaining unit, a volume information calculating unit, and a controlling unit. The image obtaining unit obtains a plurality of groups of two-dimensional ultrasound image data each of which is generated by performing ultrasound scans, with the ultrasound scans being performed on each of a plurality of predetermined cross-sectional planes, and with the ultrasound scans being performed for predetermined time periods equal to or longer than one heartbeat. The contour position obtaining unit obtains, by performing a tracking process including a two-dimensional pattern matching process over the predetermined time period, time-series data of contour positions, the contour positions being either one of, or both of, a cavity interior and a cavity exterior of a predetermined site included in each of the plurality of groups of two-dimensional ultrasound image data. The volume information calculating unit calculates, on a basis of a plurality of the time-series data of contour positions, volume information of the predetermined site, with each of the time-series data being obtained from each of the plurality of groups of the two-dimensional ultrasound image data. The controlling unit exercises control so as to output the volume information.

Exemplary embodiments of an ultrasound diagnosis apparatus will be explained in detail below, with reference to the accompanying drawings.

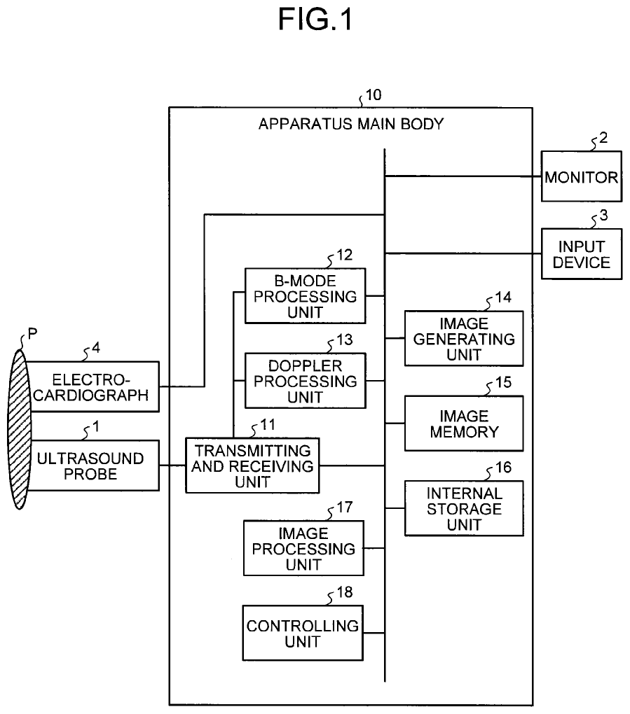

First, a configuration of an ultrasound diagnosis apparatus according to a first embodiment will be explained. FIG. 1 is a block diagram of an exemplary configuration of the ultrasound diagnosis apparatus according to the first embodiment. As shown in FIG. 1, the ultrasound diagnosis apparatus according to the first embodiment includes an ultrasound probe 1, a monitor 2, an input device 3, an electrocardiograph 4, and an apparatus main body 10.

The ultrasound probe 1 includes a plurality of piezoelectric transducer elements, which generate an ultrasound wave based on a drive signal supplied from a transmitting and receiving unit 11 included in the apparatus main body 10 (explained later). Furthermore, the ultrasound probe 1 receives a reflected wave from an examined subject P and converts the received reflected wave into an electric signal. Furthermore, the ultrasound probe 1 includes matching layers included in the piezoelectric transducer elements, as well as a backing member that prevents ultrasound waves from propagating rearward from the piezoelectric transducer elements. The ultrasound probe 1 is detachably connected to the apparatus main body 10.

When an ultrasound wave is transmitted from the ultrasound probe 1 to the subject P, the transmitted ultrasound wave is repeatedly reflected on a surface of discontinuity of acoustic impedances at a tissue in the body of the subject P and is received as a reflected-wave signal by the plurality of piezoelectric transducer elements included in the ultrasound probe 1. The amplitude of the received reflected-wave signal is dependent on the difference between the acoustic impedances on the surface of discontinuity on which the ultrasound wave is reflected. When the transmitted ultrasound pulse is reflected on the surface of a flowing bloodstream or a cardiac wall, the reflected-wave signal is, due to the Doppler effect, subject to a frequency shift, depending on a velocity component of the moving members with respect to the ultrasound wave transmission direction.

The ultrasound probe 1 used in the first embodiment is configured to scan the subject P two-dimensionally, while using the ultrasound waves. For example, the ultrasound probe 1 is a one-dimensional (1D) array probe in which a plurality of piezoelectric transducer elements are arranged in a row. However, the ultrasound probe 1 according to the first embodiment may be, for example, a mechanical four-dimensional (4D) probe or a two-dimensional (2D) array probe that is able to, while using the ultrasound waves, scan the subject P two-dimensionally and is also able to scan the subject P three-dimensionally. The mechanical 4D probe is able to perform a two-dimensional scan by employing a plurality of piezoelectric transducer elements arranged in a row and is also able to perform a three-dimensional scan by causing a plurality of piezoelectric transducer elements arranged in a row to swing at a predetermined angle (a swinging angle). The 2D array probe is able to perform a three-dimensional scan by employing a plurality of piezoelectric transducer elements arranged in a matrix formation and is also able to perform a two-dimensional scan by transmitting ultrasound waves in a focused manner. Furthermore, the 2D array probe is also able to perform two-dimensional scans on a plurality of cross-sectional planes at the same time.

The input device 3 includes a mouse, a keyboard, a button, a panel switch, a touch command screen, a foot switch, a trackball, a joystick, and the like. The input device 3 receives various types of setting requests from an operator of the ultrasound diagnosis apparatus and transfers the received various types of setting requests to the apparatus main body 10. Setting information received from the operator by the input device 3 according to the first embodiment will be explained in detail later.

The monitor 2 displays a Graphical User Interface (GUI) used by the operator of the ultrasound diagnosis apparatus to input the various types of setting requests through the input device 3 and displays an ultrasound image and the like generated by the apparatus main body 10. To inform the operator of a processing status of the apparatus main body 10, the monitor 2 displays various types of messages.

Furthermore, the monitor 2 has a speaker and is also able to output audio. For example, to inform the operator of a processing status of the apparatus main body 10, the speaker of the monitor 2 outputs predetermined audio such as a beep.

The electrocardiograph 4 is configured to obtain an electrocardiogram (ECG) of the subject P, as a biological signal of the subject P who is two-dimensionally scanned. The electrocardiograph 4 transmits the obtained electrocardiogram to the apparatus main body 10.

The apparatus main body 10 is an apparatus that generates ultrasound image data based on the reflected-wave signal received by the ultrasound probe 1. The apparatus main body 10 shown in FIG. 1 is an apparatus configured to be able to generate two-dimensional ultrasound image data, based on two-dimensional reflected-wave data received by the ultrasound probe 1.

As shown in FIG. 1, the apparatus main body 10 includes the transmitting and receiving unit 11, a B-mode processing unit 12, a Doppler processing unit 13, an image generating unit 14, an image memory 15, an internal storage unit 16, an image processing unit 17, and a controlling unit 18.

The transmitting and receiving unit 11 includes a pulse generator, a transmission delaying unit, a pulser, and the like and supplies the drive signal to the ultrasound probe 1. The pulse generator repeatedly generates a rate pulse for forming a transmission ultrasound wave at a predetermined rate frequency. Furthermore, the transmission delaying unit applies a delay period that is required to focus the ultrasound wave generated by the ultrasound probe 1 into the form of a beam and to determine transmission directionality and that corresponds to each of the piezoelectric transducer elements, to each of the rate pulses generated by the pulse generator. Furthermore, the pulser applies a drive signal (a drive pulse) to the ultrasound probe 1 with timing based on the rate pulses. In other words, the transmission delaying unit arbitrarily adjusts the transmission directions of the ultrasound waves transmitted from the piezoelectric transducer element surfaces, by varying the delay periods applied to the rate pulses.

The transmitting and receiving unit 11 has a function to be able to instantly change the transmission frequency, the transmission drive voltage, and the like, for the purpose of executing a predetermined scanning sequence based on an instruction from the controlling unit 18 (explained later). In particular, the configuration to change the transmission drive voltage is realized by using a linear-amplifier-type transmitting circuit of which the value can be instantly switched or by using a mechanism configured to electrically switch between a plurality of power source units.

The transmitting and receiving unit 11 includes a pre-amplifier, an Analog/Digital (A/D) converter, a reception delaying unit, an adder, and the like and generates reflected-wave data by performing various types of processes on the reflected-wave signal received by the ultrasound probe 1. The pre-amplifier amplifies the reflected-wave signal for each of channels. The A/D converter applies an A/D conversion to the amplified reflected-wave signal. The reception delaying unit applies a delay period required to determine reception directionality to the result of the A/D conversion. The adder performs an adding process on the reflected-wave signals processed by the reception delaying unit so as to generate the reflected-wave data. As a result of the adding process performed by the adder, reflected components from the direction corresponding to the reception directionality of the reflected-wave signals are emphasized. A comprehensive beam used in an ultrasound transmission/reception is thus formed according to the reception directionality and the transmission directionality.

When a two-dimensional scan is performed on the subject P, the transmitting and receiving unit 11 causes the ultrasound probe 1 to transmit two-dimensional ultrasound beams. The transmitting and receiving unit 11 then generates the two-dimensional reflected-wave data from the two-dimensional reflected-wave signals received by the ultrasound probe 1.

Output signals from the transmitting and receiving unit 11 can be in a form selected from various forms. For example, the output signals may be in the form of signals called Radio Frequency (RF) signals that contain phase information or may be in the form of amplitude information obtained after an envelope detection process.

The B-mode processing unit 12 receives the reflected-wave data from the transmitting and receiving unit 11 and generates data (B-mode data) in which the strength of each signal is expressed by a degree of brightness, by performing a logarithmic amplification, an envelope detection process, and the like on the received reflected-wave data.

The Doppler processing unit 13 extracts bloodstreams, tissues, and contrast echo components under the influence of the Doppler effect by performing a frequency analysis so as to obtain velocity information from the reflected-wave data received from the transmitting and receiving unit 11, and further generates data (Doppler data) obtained by extracting moving member information such as a velocity, a dispersion, a power, and the like for a plurality of points.

The B-mode processing unit 12 and the Doppler processing unit 13 shown in FIG. 1 are able to process both two-dimensional reflected-wave data and three-dimensional reflected-wave data. In other words, the B-mode processing unit 12 is able to generate two-dimensional B-mode data from the two-dimensional reflected-wave data and to generate three-dimensional B-mode data from three-dimensional reflected-wave data. The Doppler processing unit 13 is able to generate two-dimensional Doppler data from the two-dimensional reflected-wave data and to generate three-dimensional Doppler data from three-dimensional reflected-wave data.

The image generating unit 14 generates ultrasound image data from the data generated by the B-mode processing unit 12 and the Doppler processing unit 13. In other words, from the two-dimensional B-mode data generated by the B-mode processing unit 12, the image generating unit 14 generates two-dimensional B-mode image data in which the strength of the reflected wave is expressed by a degree of brightness. Furthermore, from the two-dimensional Doppler data generated by the Doppler processing unit 13, the image generating unit 14 generates two-dimensional Doppler image data expressing moving member information. The two-dimensional Doppler image data is a velocity image, a dispersion image, a power image, or an image combining these images. Furthermore, the image generating unit 14 is also able to generate M-mode image data from time-series data of the B-mode data obtained on one scanning line and generated by the B-mode processing unit 12. Furthermore, from the Doppler data generated by the Doppler processing unit 13, the image generating unit 14 is also able to generate a Doppler waveform in which velocity information of bloodstream or a tissue is plotted along a time series.

In this situation, generally speaking, the image generating unit 14 converts (by performing a scan convert process) a scanning line signal sequence from an ultrasound scan into a scanning line signal sequence in a video format used by, for example, television and generates display-purpose ultrasound image data. More specifically, the image generating unit 14 generates the display-purpose ultrasound image data by performing a coordinate transformation process compliant with the ultrasound scanning mode used by the ultrasound probe 1. Furthermore, as various types of image processing processes other than the scan convert process, the image generating unit 14 performs, for example, an image processing process (a smoothing process) to re-generate a luminance-average image or an image processing process (an edge enhancement process) using a differential filter within images, while using a plurality of image frames obtained after the scan convert process is performed. Furthermore, the image generating unit 14 synthesizes text information of various parameters, scale graduations, body marks, and the like with the ultrasound image data.

In other words, the B-mode data and the Doppler data are the ultrasound image data before the scan convert process is performed. The data generated by the image generating unit 14 is the display-purpose ultrasound image data obtained after the scan convert process is performed. The B-mode data and the Doppler data may also be referred to as raw data. The image generating unit 14 generates “two-dimensional B-mode image data or two-dimensional Doppler image data”, which is display-purpose two-dimensional ultrasound image data, from “two-dimensional B-mode data or two-dimensional Doppler data”, which is the two-dimensional ultrasound image data before the scan convert process is performed.

The image memory 15 is a memory for storing therein the display-purpose image data generated by the image generating unit 14. Furthermore, the image memory 15 is also able to store therein the data generated by the B-mode processing unit 12 and the Doppler processing unit 13. After a diagnosis process, for example, the operator is able to invoke the B-mode data or the Doppler data stored in the image memory 15. The invoked data serves as the display-purpose ultrasound image data via the image generating unit 14.

The image generating unit 14 stores, into the image memory 15, the ultrasound image data and the time at which an ultrasound scan was performed to generate the ultrasound image data, while keeping the data and the time in correspondence with an electrocardiogram transmitted from the electrocardiograph 4. By referring to the data stored in the image memory 15, the image processing unit 17 and the controlling unit 18 (explained later) are able to obtain a cardiac phase during the ultrasound scan that was performed to generate the ultrasound image data.

The internal storage unit 16 stores therein various types of data such as a control computer program (hereinafter, “control program”) to realize ultrasound transmissions and receptions, image processing, and display processing, as well as diagnosis information (e.g., patients' IDs, medical doctors' observations), diagnosis protocols, and various types of body marks. Furthermore, the internal storage unit 16 may be used, if necessary, for storing therein any of the image data stored in the image memory 15. Furthermore, it is possible to transfer the data stored in the internal storage unit 16 to external apparatuses via an interface (not shown). Examples of the external apparatuses include a personal computer (PC) used by a medical doctor who performs an image diagnosis process, storage media such as Compact Disks (CDs) and Digital Versatile Disks (DVDs), printers, and the like.

The image processing unit 17 is provided in the apparatus main body 10 to perform a computer-aided diagnosis (CAD) process. The image processing unit 17 obtains the ultrasound image data stored in the image memory 15 and performs image processing processes to aid diagnosis processes. After that, the image processing unit 17 stores results of the image processing processes into the image memory 15 and/or the internal storage unit 16. Processes performed by the image processing unit 17 will be explained in detail later.

The controlling unit 18 is configured to control the entire processes performed by the ultrasound diagnosis apparatus. More specifically, based on the various types of setting requests input by the operator via the input device 3 and various types of control programs and various types of data read from the internal storage unit 16, the controlling unit 18 controls processes performed by the transmitting and receiving unit 11, the B-mode processing unit 12, the Doppler processing unit 13, the image generating unit 14, and the image processing unit 17. Furthermore, the controlling unit 18 exercises control so that the monitor 2 displays the display-purpose ultrasound image data stored in the image memory 15 and the internal storage unit 16. Furthermore, the controlling unit 18 exercises control so that processing results from the image processing unit 17 are displayed on the monitor 2 or are output to external apparatuses. Furthermore, the controlling unit 18 exercises control so that predetermined audio is output from the speaker of the monitor 2, on the basis of the processing results from the image processing unit 17.

An overall configuration of the ultrasound diagnosis apparatus according to the first embodiment has thus been explained. The ultrasound diagnosis apparatus according to the first embodiment configured as described above measures volume information by using the two-dimensional ultrasound image data. For example, the ultrasound diagnosis apparatus according to the first embodiment measures volume information of the heart, by using two-dimensional ultrasound image data generated by performing an ultrasound scan on a cross-sectional plane containing the heart of the subject P.

Conventionally, during an echocardiography, volume information of the heart is mainly estimated by using the M-mode method, for reasons of convenience; however, there are some situations where volume information estimated by using the M-mode method contains an error. To cope with these situations, a method that uses two-dimensional ultrasound image data (two-dimensional B-mode image data) is known as a method by which it is possible to estimate volume information with an excellent level of precision. In the following sections, the method for estimating the volume information by using the two-dimensional ultrasound image data will be explained.

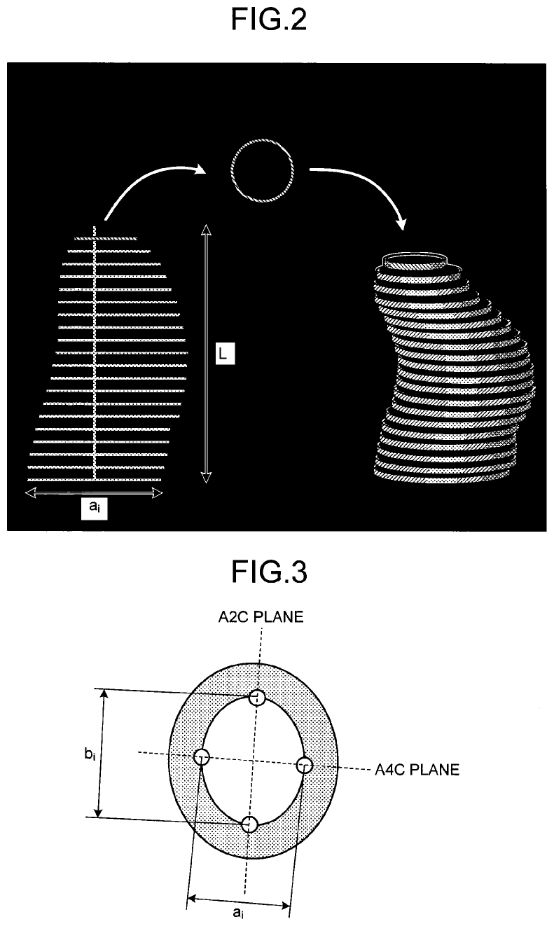

An “area-length method” and a “disc summation method (a Simpson's method)” are known as methods for estimating volume information with an excellent level of precision by which a three-dimensional shape of a cavity interior (i.e., a lumen) is estimated on the basis of a two-dimensional contour rendered in two-dimensional ultrasound image data taken on one cross-sectional plane. FIG. 2 is a drawing for explaining the disc summation method (the Simpson's method).

When implementing the disc summation method (the Simpson's method), for example, a conventional ultrasound diagnosis apparatus receives a setting of a cavity interior region (a contour position of the cavity interior) on the basis of information resulting from the operator's tracing the contour of the left ventricular cavity interior rendered in an A4C view and further detects a long axis of the cavity interior region that was set. Alternatively, the operator may set two points for specifying the long axis. Furthermore, as shown in FIG. 2, for example, the conventional ultrasound diagnosis apparatus equally divides the left ventricular cavity interior region set in the A4C view into twenty discs that are perpendicular to the long axis (see “L” in FIG. 2) of the left ventricle. After that, the conventional ultrasound diagnosis apparatus calculates a distance (see aᵢ in FIG. 2) between the two points at which an i'th disc intersects the inner layer surface. Subsequently, as shown in FIG. 2, the conventional ultrasound diagnosis apparatus approximates a three-dimensional shape of the cavity interior of the i'th disc as a slice of a cylinder having the diameter “aᵢ”. Furthermore, the conventional ultrasound diagnosis apparatus calculates a summation of the volumes of the twenty discs as volume information approximating the volume of the cavity interior, by using Expression (1) below. In Expression (1), the length of the long axis is expressed as “L”.

$$V = \frac{π}{4} {\sum }_{i = 1}^{20} {a}_{i}^{2} \cdot \frac{L}{20} \qquad ( 1 )$$

In contrast, the “area-length method” is a method by which, for example, while the left ventricle is assumed to be a spheroid, an approximate value of the volume of the cavity interior is calculated by calculating the length of the short axis of the left ventricular cavity interior, on the basis of measured results of a left ventricular cavity interior area containing the long axis (L) of the left ventricle and the length of the long axis of the left ventricular cavity interior. When implementing the “area-length method”, for example, a conventional ultrasound diagnosis apparatus calculates volume information approximating the volume of the cavity interior with the expression “8×(cavity interior area)²/(3×π×L)”, while using the left ventricular cavity interior area and the length “L” of the long axis of the left ventricular cavity interior based on the tracing process performed by the operator.

Furthermore, as a method for estimating volume information with an even higher level of precision than the “area-length method” and the “disc summation method (the Simpson's method)”, a “modified-Simpson's method” is known, which is a method obtained by modifying the “disc summation method (the Simpson's method)”. FIG. 3 is a drawing for explaining the modified-Simpson's method.

According to the “modified-Simpson's method”, for example, an A4C view and an A2C view acquired by performing a two-dimensional scan on each of two cross-sectional planes such as an A4C plane and an A2C plane are used. When implementing the “modified-Simpson's method”, for example, a conventional ultrasound diagnosis apparatus receives a setting of a cavity interior region (a contour position of the cavity interior) on the basis of information resulting from the operator's tracing the contour of the left ventricular cavity interior rendered in the A4C view and further detects a long axis of the cavity interior region that was set. In addition, the conventional ultrasound diagnosis apparatus receives a setting of a cavity interior region (a contour position of the cavity interior) on the basis of, for example, the operator's tracing the contour of the left ventricular cavity interior rendered in the A2C view and further detects a long axis of the cavity interior region that was set. Alternatively, the operator may set two points for specifying the long axis on each of the cross-sectional planes. Furthermore, for example, the conventional ultrasound diagnosis apparatus equally divides each of the A4C and the A2C views into twenty discs that are perpendicular to the long axis. After that, as shown in FIG. 3 for example, the conventional ultrasound diagnosis apparatus calculates the distance (see aᵢ in FIG. 3) between the two points at which an i'th disc on the A4C plane intersects the inner layer surface, as well as the distance (see bᵢ in FIG. 3) between the two points at which an i'th disc on the A2C plane intersects the inner layer surface. Subsequently, the conventional ultrasound diagnosis apparatus approximates a three-dimensional shape of the cavity interior of the i'th disc as a slice of an ellipsoid having a long axis and a short axis estimated from “aᵢ” and “bᵢ”. Furthermore, the conventional ultrasound diagnosis apparatus calculates a summation of the volumes of the twenty discs as volume information approximating the volume of the cavity interior, by using Expression (2) below. In Expression (2), a representative value (e.g., a maximum value or an average value) calculated from the length of the long axis in the A4C view and the length of the long axis in the A2C view is expressed as “L”.

$$V = \frac{π}{4} {\sum }_{i = 1}^{20} ( {a}_{i} \cdot {b}_{i} ) \frac{L}{20} \qquad ( 2 )$$

Furthermore, for the “area-length method” also, a method (a “biplane area-length method”) used for improving the level of precision in the estimation of the volume of the cavity interior has conventionally been reported, by which measured results on two mutually-different cross-sectional planes (e.g., an A4C view and an A2C view) are used. According to the “biplane area-length method”, volume information is calculated by approximating the volume of the cavity interior with the expression “8×(cavity interior area on cross-sectional plane 1)×(cavity interior area on cross-sectional plane 2)/(3×π×L) where L is the longer of the lengths of the long axis between cross-sectional plane 1 and cross-sectional plane 2”. In the following sections, from among various methods using two cross-sectional planes, the “modified-Simpson's method” will be used in the explanation as an example.

When the “modified-Simpson's method” is used, if an error in the lengths of the long axis on the two cross-sectional planes is equal to or larger than 20%, it is necessary to perform the measuring process again. However, if an error in the lengths of the long axis on the two cross-sectional planes is equal to or smaller than 10%, the level of precision of the volume information measuring process using the “modified-Simpson's method” is known to be sufficiently high in practice, even with medical cases exhibiting a regional wall motion abnormality (e.g., medical cases where the shape of the cavity interior is complicated).

In this situation, examples of volume information of a ventricle or an atrium of the heart include a volume of the cavity interior, a myocardial volume calculated from a cavity exterior volume and a cavity interior volume, and a myocardial mass calculated from a myocardial volume. Furthermore, in particular, examples of volume information that are important when making diagnoses of cardiac diseases include EF values (an ejection fraction (EF) for the left ventricle and an empty fraction (EF) for the left atrium), each of which is an index value indicating the pumping function of the ventricle or the atrium. The EF value is a value defined by a volume of the cavity interior at an end diastole (ED) and a volume of the cavity interior at an end systole (ES).

When the volume information described above is measured by using the “modified-Simpson's method”, a process that is manually performed by the operator includes the following five steps:

For example, the operator first acquires two-dimensional ultrasound image data of A4C views along a time series. After that, the operator acquires two-dimensional ultrasound image data of A2C views along a time series. As a result, the operator has obtained moving image data of the A4C views (hereinafter, a “group of A4C views”) and moving image data of the A2C views (hereinafter, a “group of A2C views”) (the first step).

Subsequently, the operator selects an A4C view at the ED out of the group of A4C views and traces the cavity interior (the inner layer of the myocardium) rendered in the selected A4C view at the ED (the second step). In this situation, if the operator wishes to obtain a volume of the cavity exterior as volume information, the operator also traces the cavity exterior (the outer layer of the myocardium) rendered in the A4C view at the ED.

After that, the operator selects an A4C view in an ES time phase out of the group of A4C views and traces the cavity interior rendered in the selected A4C view in the ES time phase (the third step). In this situation, if the operator wishes to obtain a volume of the cavity exterior as volume information, the operator also traces the cavity exterior rendered in the A4C view in the ES time phase.

After that, the operator selects an A2C view at the ED out of the group of A2C views and traces the cavity interior rendered in the selected A2C view at the ED (the fourth step). In this situation, if the operator wishes to obtain a volume of the cavity exterior as volume information, the operator also traces the cavity exterior rendered in the A2C view at the ED.

Subsequently, the operator selects an A2C view at the ES out of the group of A2C views and traces the cavity interior rendered in the selected A2C view at the ES (the fifth step). In this situation, if the operator wishes to obtain a volume of the cavity exterior as volume information, the operator also traces the cavity exterior rendered in the A2C view at the ES.

After receiving the five steps described above, a conventional ultrasound diagnosis apparatus implements the “modified-Simpson's method” and outputs a measured result (an estimated result) of volume information. However, it is cumbersome for the operator to manually perform the five steps described above, and this process requires a lot of labor from the operator. For this reason, the “modified-Simpson's method” is not widely used in the actual clinical field. Also, when the “biplane area-length method” is implemented, the five steps described above are manually performed by the operator. Thus, the “biplane area-length method” is not a method that allows the operator to easily obtain the volume information, either.

To cope with this situation, the ultrasound diagnosis apparatus according to the first embodiment causes the image processing unit 17 to perform processes described below, for the purpose of easily obtaining a measured result of volume information with a high level of precision.

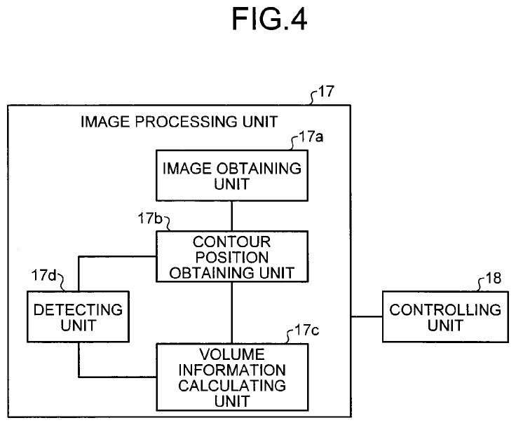

FIG. 4 is a block diagram of an exemplary configuration of the image processing unit according to the first embodiment. As shown in FIG. 4, the image processing unit 17 according to the first embodiment includes an image obtaining unit 17a, a contour position obtaining unit 17b, a volume information calculating unit 17c, and a detecting unit 17d.

In the first embodiment, by using the ultrasound probe 1, the operator first performs an ultrasound scan on each of a plurality of predetermined cross-sectional planes for a predetermined time period equal to or longer than one heartbeat. For example, to acquire A4C views, which are long-axis views of the heart, along a time series, the operator performs an ultrasound scan on an A4C plane for a time period equal to or longer than one heartbeat, while taking an apex approach. As a result, the image generating unit 14 generates a plurality of pieces of two-dimensional ultrasound image data on the A4C plane along the time series for the predetermined time period and stores the generated data into the image memory 15. In addition, to acquire A2C views, which are long-axis views of the heart, along a time series, the operator performs an ultrasound scan on an A2C plane for a predetermined time period equal to or longer than one heartbeat, while taking an apex approach. As a result, the image generating unit 14 generates a plurality of pieces of two-dimensional ultrasound image data (A2C views) on the A2C plane along the time series for the predetermined time period and stores the generated data into the image memory 15. The two-dimensional ultrasound image data in the first embodiment is two-dimensional B-mode image data.

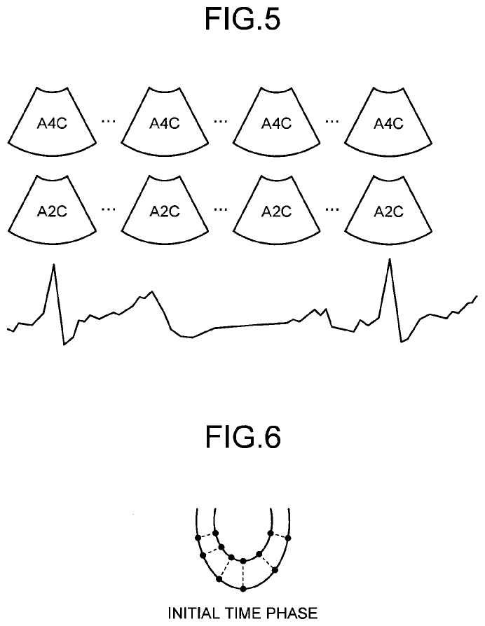

After that, the image obtaining unit 17a obtains a plurality of groups of two-dimensional ultrasound image data each of which is generated by performing the ultrasound scan on each one of the plurality of predetermined cross-sectional planes for a predetermined time period equal to or longer than one heartbeat. FIG. 5 is a drawing for explaining the image obtaining unit according to the first embodiment. As shown in FIG. 3 for example, the image obtaining unit 17a obtains a plurality of pieces of two-dimensional ultrasound image data (a group of A4C views) on the A4C plane along the time series for a one-heartbeat period, as well as a plurality of pieces of two-dimensional ultrasound image data (a group of A2C views) on the A2C plane along the time series for a one-heartbeat period. In this situation, the image obtaining unit 17a obtains the group of A4C views for the one-heartbeat period and the group of A2C views for the one-heartbeat period by detecting a time phase having a characteristic wave (e.g., an R-wave or a P-wave) from the electrocardiogram obtained by the electrocardiograph 4.

After that, the contour position obtaining unit 17b shown in FIG. 4 obtains time-series data of contour positions of one or both of the cavity interior and the cavity exterior of the predetermined site included in each of the plurality of groups of two-dimensional ultrasound image data, by performing a tracking process including a two-dimensional pattern matching process over the predetermined time period. In other words, the contour position obtaining unit 17b performs a two-dimensional speckle tracking (2DT) process on the two-dimensional moving image data. The speckle tracking method is a method by which an accurate motion is estimated by using, for example, an optical flow method or other various spatio-temporal interpolation processes, together with the pattern matching process. Examples of the speckle tracking method include a method by which a motion is estimated without performing the pattern matching process.

In this situation, the contour position obtaining unit 17b obtains contour positions of at least one of the ventricles and the atria of the heart as the predetermined site. In other words, the operator selects one or more sites as a target of the 2DT process from among the following: the cavity interior of the right atrium; the cavity exterior of the right atrium, the cavity interior of the right ventricle; the cavity exterior of the right ventricle; the cavity interior of the left atrium; the cavity exterior of the left atrium, the cavity interior of the left ventricle; and the cavity exterior of the left ventricle. In the following sections, an example will be explained in which the cavity interior of the left ventricle and the cavity exterior of the left ventricle are selected as the sites serving as the target of the 2DT process.

For example, the input device 3 receives a tracking point setting request from the operator. When the tracking point setting request is transferred to the controlling unit 18, the controlling unit 18 reads two-dimensional ultrasound image data in an initial time phase from the image memory 15 and causes the monitor 2 to display the read image data.

More specifically, the controlling unit 18 uses an ED as the initial time phase, reads an A4C view at the ED and an A2C view at the ED from the image memory 15, and causes the monitor 2 to display the read views. For example, the controlling unit 18 selects an A4C view in an R-wave time phase out of the moving image data of the A4C views, as the A4C view at the ED. In addition, for example, the controlling unit 18 selects an A2C view in an R-wave time phase out of the moving image data of the A2C views, as the A2C view at the ED.

Alternatively, the controlling unit 18 may use an ES as the initial time phase, may read an A4C view at the ES and an A2C view at the ES from the image memory 15, and may cause the monitor 2 to display the read views. When an ES is used as the initial time phase, the controlling unit 18 refers to a table that is stored in advance, selects an A4C view at the ES out of the moving image data of the A4C views, and selects an A2C view at the ES out of the moving image data of the A2C views. For example, as the table used for estimating two-dimensional ultrasound image data corresponding to the ES time phase, the internal storage unit 16 stores therein a table in which elapsed time periods between a reference time phase (e.g., an R-wave time phase) and an ES time phase are kept in correspondence with heart rates. The controlling unit 18 calculates a heart rate from the electrocardiogram of the subject P and obtains an elapsed time period corresponding to the calculated heart rate. After that, the controlling unit 18 selects two-dimensional ultrasound image data corresponding to the obtained elapsed time period out of the moving image data and causes the monitor 2 to display the selected two-dimensional ultrasound image data as the two-dimensional ultrasound image data at the ES.

The process to select the data in the initial time phase may be performed by, for example, the image obtaining unit 17a or the contour position obtaining unit 17b, instead of the controlling unit 18. Furthermore, the first frame in the moving image data may be used as the initial time phase.

FIG. 6 is a drawing for explaining an example of the two-dimensional speckle tracking process. The operator sets tracking points at which a 2DT process is to be performed, by referring to the two-dimensional ultrasound image data in the initial time phase shown in FIG. 6. For example, the operator traces the inner layer of the left ventricle and the outer layer of the left ventricle in the two-dimensional ultrasound image data in the initial time phase, by using the mouse of the input device 3. The contour position obtaining unit 17b reconstructs two two-dimensional boundary planes from the traced inner layer surface and the traced outer layer surface, as two contours in the initial time phase (initial contours). Furthermore, as shown in FIG. 6, the contour position obtaining unit 17b sets a plurality of tracking points in pairs on the inner layer surface and the outer layer surface in the initial time phase. The contour position obtaining unit 17b sets template data with each of the plurality of tracking points that were set in a frame in the initial time phase. The template data is structured with a plurality of pixels centered on each of the tracking points.

Furthermore, the contour position obtaining unit 17b tracks the template data to find out the position to which the template data has moved in the subsequent frame, by searching for a region that best matches the speckle pattern of the template data between the two frames. By performing the tracking process in this manner, the contour position obtaining unit 17b obtains the positions of the tracking points in the group of two-dimensional ultrasound image data other than the two-dimensional ultrasound image data in the initial time phase.

As a result, the contour position obtaining unit 17b obtains, for example, time-series data of the contour positions of the left ventricular cavity interior included in the A4C views and time-series data of the contour positions of the left ventricular cavity exterior included in the A4C views. Furthermore, for example, the contour position obtaining unit 17b obtains time-series data of the contour positions of the left ventricular cavity interior included in the A2C views and time-series data of the contour positions of the left ventricular cavity exterior included in the A2C views. As a result of the contour position obtaining unit 17b performing the 2DT process as described above, the third and the fifth steps in the conventional example described above or the second and the fourth steps in the conventional example described above are automated.

The initial contour setting process does not necessarily have to be manually performed by the operator as described above. For example, the initial contour setting process may be automatically performed as described below. For example, the contour position obtaining unit 17b estimates a position of the initial contour, on the basis of a position of the annulus site and a position of the apex site that are specified by the operator in the image data in the initial time phase. Alternatively, for example, the contour position obtaining unit 17b estimates a position of the initial contour from the image data in the initial time phase, without receiving any information from the operator. These automatic estimating processes are performed by using a boundary estimating technique that utilizes brightness information of the image or another boundary estimating technique that estimates a boundary by comparing features of the image with a shape dictionary that has registered therein in advance “shape information of the heart”, while using a discriminator. When the initial contour setting process is automatically performed, the second to the fifth steps in the conventional example described above are automated.

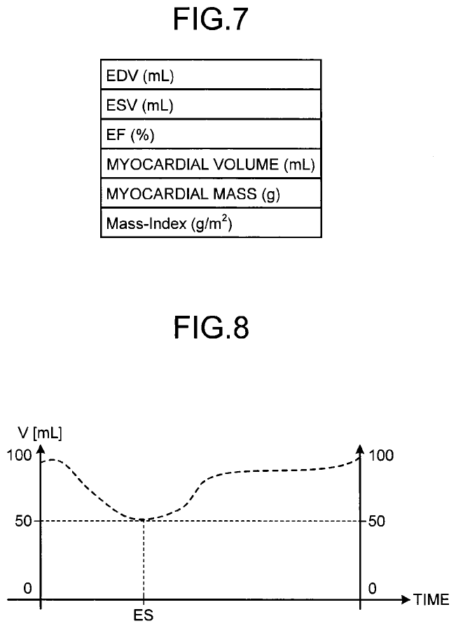

The volume information calculating unit 17c shown in FIG. 4 is configured to calculate volume information of a predetermined site, on the basis of the pieces of the plurality of time-series data of contour positions each of which was obtained from a different one of the plurality of groups of two-dimensional ultrasound image data. More specifically, the volume information calculating unit 17c calculates the volume information by using the “modified-Simpson's method”, which is a method obtained by modifying the disc summation method that estimates a volume from two-dimensional image data on a plurality of cross-sectional planes. FIG. 7 is a table of examples of volume information calculated by the volume information calculating unit according to the first embodiment.

As shown in FIG. 7, the volume information calculating unit 17c according to the first embodiment calculates at least one of the following as the volume information: numerical information about an end-diastolic volume “EDV (ml)”; numerical information about an end-systolic volume “ESV (ml)”; numerical information about an ejection fraction “EF (%)”; numerical information about a myocardial volume (mL); numerical information about a myocardial mass (g); and numerical information about a mass-index (g/m²).

For example, the volume information calculating unit 17c calculates an EDV of the left ventricle by using the “modified Simpson's method” explained above, on the basis of the contour position at the ED in the time-series data of the contour positions of the left ventricular cavity interior in the A4C views and the contour position at the ED in the time-series data of the contour positions of the left ventricular cavity interior in the A2C views. Furthermore, the volume information calculating unit 17c calculates an ESV of the left ventricle by using the “modified Simpson's method” explained above, on the basis of the contour position at the ES in the time-series data of the contour positions of the left ventricular cavity interior in the A4C views and the contour position at the ES in the time-series data of the contour positions of the left ventricular cavity interior in the A2C views. After that, the volume information calculating unit 17c calculates an ejection fraction of the left ventricle from the EDV of the left ventricle and the ESV of the left ventricle.

Furthermore, the volume information calculating unit 17c calculates a volume of the left ventricular cavity exterior at the ED by using the “modified Simpson's method” explained above, on the basis of the contour position at the ED in the time-series data of the contour positions of the left ventricular cavity exterior in the A4C views and the contour position at the ED in the time-series data of the contour positions of the left ventricular cavity exterior in the A2C views. After that, by subtracting the EDV from the volume of the left ventricular cavity exterior at the ED, the volume information calculating unit 17c calculates a myocardial volume. In this situation, although myocardial volumes change in accordance with heartbeats, the degree by which myocardial volumes change over the course of time is small. Thus, it is possible to use a specific cardiac phase (e.g., an ED) as the time phase for calculating the volume of the cavity exterior. It is also acceptable to use a time phase (e.g., an ES) other than the ED, as the time phase for calculating the volume of the cavity exterior.

Furthermore, the volume information calculating unit 17c calculates the “myocardial mass (g)” by multiplying the “myocardial volume (mL)” by an average myocardial density value (e.g., 1.05 g/mL). Furthermore, the volume information calculating unit 17c calculates the “mass-index (g/m²)” by normalizing the “myocardial mass (g)” with a “body surface area (BSA) (m²)”. It is also acceptable if the volume information calculating unit 17c according to the first embodiment calculates the volume information by using the “biplane area-length method”, which is a method obtained by modifying the “area-length method”.

In this situation, the volume information calculating unit 17c is able to obtain the contour position in the ED phase, by selecting the contour position in the R-wave time phase as described above. To select the contour position in the ES time phase, although the volume information calculating unit 17c may use an elapsed time period obtained from the above-mentioned table, it is preferable to use one of the two selecting methods described below in order to improve the precision level in the calculation of the volume information.

The first selecting method is a method by which the operator sets an end-systolic phase. In other words, the input device 3 receives a setting for the end-systolic phase. Furthermore, on the basis of the setting information received by the input device 3, the volume information calculating unit 17c selects a contour position in the end-systolic phase from each of the pieces of the plurality of time-series data of contour positions.

More specifically, according to the first selecting method, the operator sets a time (an AVC time) at which the aortic valve of the subject P closes. It is possible to obtain the AVC time by measuring an elapsed time period from an R-wave to Sound II in the phonocardiogram, while using the R-wave as a reference. Alternatively, it is also possible to obtain the AVC time by measuring an ejection ending time from a Doppler waveform. The volume information calculating unit 17c selects the contour position in a time phase closest to the AVC time (e.g., a time phase immediately preceding the AVC time) as the contour position in the ES time phase. Although it is possible to use the first selecting method in the first embodiment, the first selecting method requires the separate measuring process to obtain the AVC time.

In contrast, the second selecting method is a method by which the contour position in the ES time phase is automatically selected, by employing the detecting unit 17d shown in FIG. 4 to automatically detect the ES time phase. The detecting unit 17d shown in FIG. 4 is configured to detect, from each of the pieces of the plurality of time-series data of contour positions, a time phase in which the volume information is the smallest or the largest, as an end-systolic phase. For example, if an atrium is the predetermined site, the detecting unit 17d detects, from each of the pieces of the plurality of time-series data of contour positions, a time phase in which the volume information is the largest, as the end-systolic phase. As another example, if a ventricle is the predetermined site, the detecting unit 17d detects, from each of the pieces of the plurality of time-series data of contour positions, a time phase in which the volume information is the smallest, as the end-systolic phase. FIG. 8 is a chart for explaining the detecting unit according to the first embodiment.

In an example, the detecting unit 17d calculates time-series data of the volume from the time-series data of contour positions on one cross-sectional plane, by using the “area-length method” or the “disc summation method” described above. For example, the detecting unit 17d calculates time-series data of the volume of the left ventricular cavity interior, by using the time-series data of the contour positions obtained by the contour position obtaining unit 17b from the moving image data of the A4C views. Furthermore, the detecting unit 17d calculates time-series data of the volume of the left ventricular cavity interior, by using the time-series data of the contour positions obtained by the contour position obtaining unit 17b from the moving image data of the A2C views. After that, as shown in FIG. 8, the detecting unit 17d detects a time phase in which the volume of the left ventricular cavity interior is the smallest in the time-series data of the volume of the left ventricular cavity interior (see the temporal change curve indicated with a broken line in FIG. 8), as an ES time phase. The detecting unit 17d may calculate time-series data of the cavity interior area from the time-series data of the contour positions as the volume information and may detect an end-systolic phase by using the time-series data of the cavity interior area. Furthermore, the volume information calculating process using the time-series data of the contour positions on one cross-sectional plane may be performed by the volume information calculating unit 17c.

After that, according to the second selecting method, the volume information calculating unit 17c selects a contour position in the end-systolic phase from each of the pieces of the plurality of time-series data, on the basis of the time phase detected by the detecting unit 17d as the end-systolic phase.

In the first embodiment, the volume information calculating unit 17c selects the contour position in the time phase that was specified as the end-systolic phase by using either the first selecting method or the second selecting method. Furthermore, by using the contour position selected as the contour position in the end-systolic phase, the volume information calculating unit 17c calculates volume information based on the end-systolic phase (e.g., a volume in the end-systolic phase, as well as an EF value based on a volume in the end-systolic phase and a volume in the end-diastolic phase).

After that, the controlling unit 18 exercises control so that the volume information calculated by the volume information calculating unit 17c is output. For example, the controlling unit 18 exercises control so that the volume information is displayed on the monitor 2. Alternatively, the controlling unit 18 exercises control so that the volume information is output to an external apparatus.

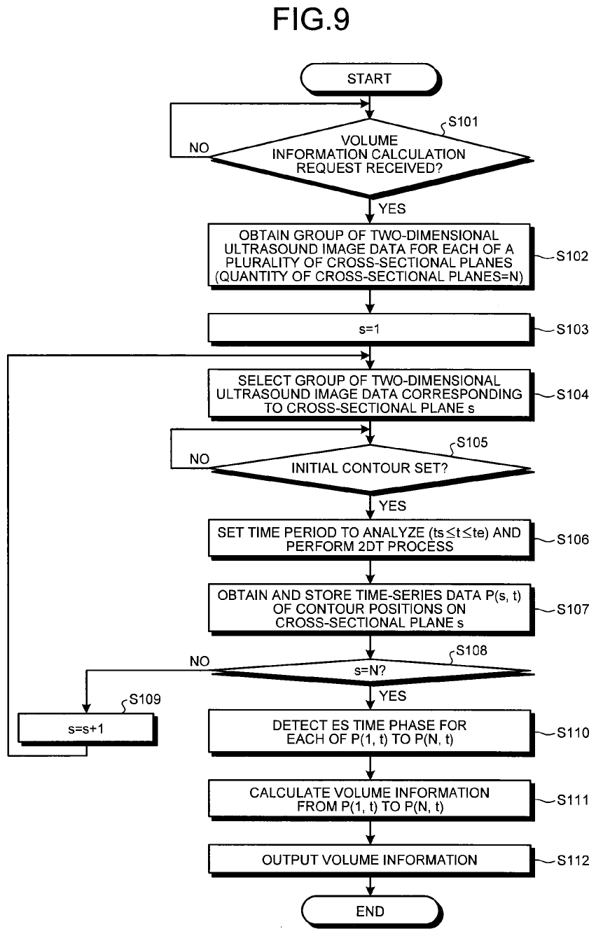

Next, a process performed by the ultrasound diagnosis apparatus according to the first embodiment will be explained with reference to FIG. 9. FIG. 9 is a flowchart for explaining an example of the process performed by the ultrasound diagnosis apparatus according to the first embodiment. FIG. 9 illustrates a flowchart in a situation where an initial contour is set by the operator, and the second selecting method employing the detecting unit 17d is implemented.

As shown in FIG. 9, the ultrasound diagnosis apparatus according to the first embodiment judges whether groups of two-dimensional ultrasound image data each corresponding to a different one of a plurality of cross-sectional planes have been specified as a processing target and whether a volume information calculation request has been received (step S101). In this situation, if a volume information calculation request has not been received (step S101: No), the ultrasound diagnosis apparatus stands by until a volume information calculation request is received.

On the contrary, if a volume information calculation request has been received (step S101: Yes), the image obtaining unit 17a obtains the specified groups of two-dimensional ultrasound image data corresponding to the plurality of cross-sectional planes (where the quantity of cross-sectional planes=N) (step S102). After that, the controlling unit 18 sets “s” so as to satisfy “s=1” (step S103), whereas the contour position obtaining unit 17b selects a group of two-dimensional ultrasound image data corresponding to a cross-sectional plane “s” (step S104). After that, the contour position obtaining unit 17b judges whether an initial contour on the cross-sectional plane “s” has been set (step S105). In this situation, if the initial contour on the cross-sectional plane “s” has not been set (step S105: No), the contour position obtaining unit 17b stands by until the initial contour is set.

On the contrary, if the initial contour has been set (step S105: Yes), the contour position obtaining unit 17b sets a time period to analyze (tste) and performs the 2DT process (step S106). For example, the contour position obtaining unit 17b performs the 2DT process by using a group of two-dimensional ultrasound image data corresponding to the cross-sectional plane “s” for a one-heartbeat period. As a result, the contour position obtaining unit 17b obtains time-series data P(s,t) of the contour positions on the cross-sectional plane “s” and stores the obtained time-series data into the internal storage unit 16 (step S107).

After that, the contour position obtaining unit 17b judges whether “s=N” is satisfied (step S108). If “s” is not equal to “N” (step S108: No), the contour position obtaining unit 17b sets “s” so as to satisfy “s=s+1” (step S109), and the process returns to step S104 where the contour position obtaining unit 17b selects a group of two-dimensional ultrasound image data on the cross-sectional plane “s”.

On the contrary, if “s=N” is satisfied (step S108: Yes), the detecting unit 17d detects an ES time phase for each of P(1,t) to P(N,t) (step S110). After that, the volume information calculating unit 17c calculates volume information on the basis of P(1,t) to P(N,t) by implementing either the “modified-Simpson's method” or the “biplane area-length” method (step S111). The controlling unit 18 exercises control so that the volume information is output (step S112), and the process ends.

As explained above, in the first embodiment, by performing the 2DT process, for example, the time-series data of the contour positions of the inner layer and of the outer layer is automatically obtained from each of the pieces of moving image data corresponding to the plurality of cross-sectional planes for the time period of at least one heartbeat. Furthermore, according to the first embodiment, it is possible to calculate the volume information (e.g., an EF value, a myocardial mass) having a high level of precision by using the automatically-obtained time-series data of the contour positions and by implementing either the “modified-Simpson's method” or the “biplane area-length” method. Thus, according to the first embodiment, it is possible to easily obtain the measured results of volume information that have a high level of precision.

Furthermore, in the first embodiment, it is possible to improve the level of convenience of the volume information calculating process by automatically detecting the ES time phase with the use of the second selecting method. In addition, it is possible to improve reproducibility of the volume information calculating process, by reducing dependency on the test administrator during the measuring process, with the use of the automatic detection.

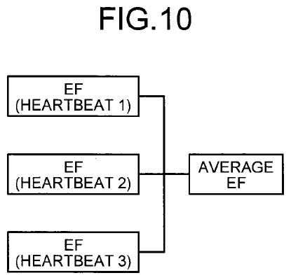

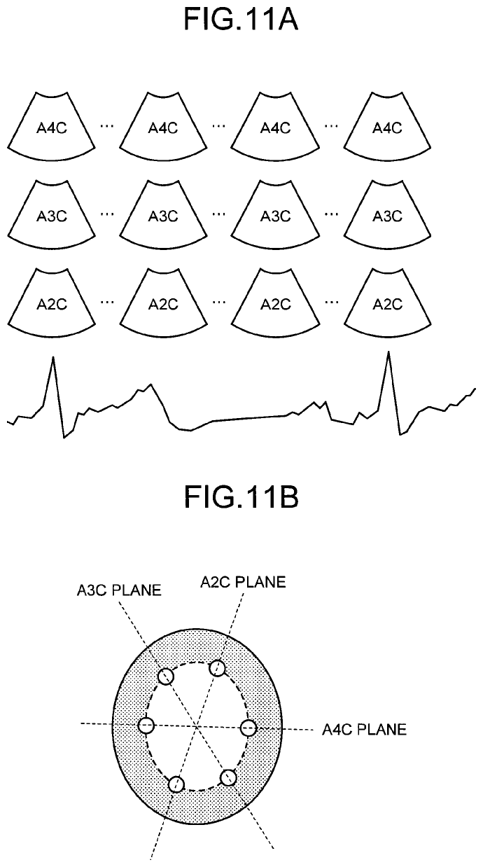

The first embodiment may be realized in the following two modification examples. The modification examples of the first embodiment will be explained with reference to FIGS. 10, 11A, and 11B. FIG. 10 is a drawing for explaining a first modification example of the first embodiment. FIGS. 11A and 11B are drawings for explaining a second modification example of the first embodiment.

In the first modification example, the contour position obtaining unit 17b obtains time-series data of contour positions corresponding to multiple heartbeats, from each of the plurality of groups of two-dimensional ultrasound image data, by performing a tracking process over the time period of the multiple consecutive heartbeats on each of the plurality of groups of two-dimensional ultrasound image data.

After that, in the first modification example, the volume information calculating unit 17c calculates volume information corresponding to the multiple heartbeats, on the basis of the pieces of time-series data of the contour positions corresponding to the multiple heartbeats, each from a different one of the plurality of groups of two-dimensional ultrasound image data. The volume information calculating unit 17c further calculates average volume information by averaging the calculated volume information corresponding to the multiple heartbeats. After that, in the first modification example, the controlling unit 18 exercises control so that the average volume information is output.

For example, as shown in FIG. 10, the volume information calculating unit 17c calculates EF (heartbeat 1), EF (heartbeat 2), and EF (heartbeat 3) as EF values corresponding to three heartbeats. Furthermore, as shown in FIG. 10, the volume information calculating unit 17c calculates an average EF value by averaging EF (heartbeat 1), EF (heartbeat 2), and EF (heartbeat 3).

In other words, it is possible to perform the 2DT process described above for a time period corresponding to multiple consecutive heartbeats. In the first modification example, the volume information corresponding to the multiple heartbeats is calculated on the basis of the result of the 2DT process performed on the multiple heartbeats, and furthermore, the pieces of volume information corresponding to the multiple heartbeats are averaged. Thus, it is possible to easily obtain stable volume information.

In the second modification example, by modifying the “modified Simpson's method” where contour information on two cross-sectional planes such as A4C views and A2C views is used, a volume is estimated from contour information on three cross-sectional planes to which contour information of an apical long-axis view (hereinafter, an “A3C view”) has further been added.

In the second modification example, the operator performs an ultrasound scan on an A4C plane, an A2C plane, and an A3C plane for a time period equal to or longer than one heartbeat. Furthermore, as shown in FIG. 11A, the image obtaining unit 17a obtains a plurality of moving image data of A4C views for the one or more heartbeats along a time series, and a plurality of moving image data of A3C views for the one or more heartbeats along the time series, as well as a plurality of moving image data of A2C views for the one or more heartbeats along the time series.

After that, the contour position obtaining unit 17b obtains time-series data of the contour positions in the A4C views, time-series data of the contour positions in the A3C views, and time-series data of the contour positions in the A2C views. Subsequently, the volume information calculating unit 17c equally divides the A4C views, the A3C views, and the A2C views each into twenty discs that are perpendicular to the long axis, on the basis of the contour positions in the A4C views, the contour positions in the A2C views, and the contour positions in the A3C views. After that, the volume information calculating unit 17c obtains positions of two points at which an i'th disc in the A4C views intersects the inner layer surface, and positions of two points at which an i'th disc in the A3C views intersects the inner layer surface, as well as positions of two points at which an i'th disc in the A2C views intersects the inner layer surface.

Subsequently, the volume information calculating unit 17c determines a shape of the cavity interior of the i'th disc on the basis of the obtained positions of the six points by performing, for example, a “spline interpolation process” (see the closed curve indicated with a broken line in FIG. 11B). After that, the volume information calculating unit 17c approximates a three-dimensional shape of the cavity interior of the i'th disc as a slice of a column that has the spline closed curve as the top face and the bottom face thereof. The volume information calculating unit 17c then calculates a summation of the volumes of the twenty columns as volume information approximating the volume of the cavity interior, by using Expression (3) below. In Expression (3), the area of the spline closed curve for an i'th disc is expressed as “Ai”. Furthermore, in Expression (3), a representative value (e.g., a maximum value or an average value) calculated from the length of the long axis in the A4C view, the length of the long axis in the A2C view, and the length of the long axis in the A3C view is expressed as “L”.

$$V = {\sum }_{i = 1}^{20} {A}_{i} \cdot \frac{L}{20} \qquad ( 3 )$$

In the second modification example, the volume information calculating unit 17c calculates and outputs the volume information obtained by using the contour positions on the three cross-sectional planes. In the second modification example, because one more cross-sectional plane is used as the processing target, the processing amount of the image processing unit 17 is increased. However, according to the second modification example, by adding the relatively simple process of adding one more scanned cross-sectional plane, it is possible to improve the level of precision in the volume measuring process in medical cases that involve a complicated shape of the heart.

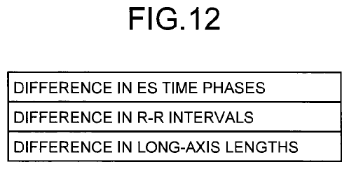

In a second embodiment, an example will be explained with reference to FIG. 12 in which the operator is informed of information that may be a cause of a decrease in the level of precision in the volume information calculating process during the automatic process explained in the first embodiment. FIG. 12 is a drawing for explaining a detecting unit according to the second embodiment.

The image processing unit 17 according to the second embodiment has the same configuration as that of the image processing unit 17 according to the first embodiment shown in FIG. 4. In other words, the image processing unit 17 according to the second embodiment includes the image obtaining unit 17a, the contour position obtaining unit 17b, the volume information calculating unit 17c, and the detecting unit 17d that are configured to perform the processes explained in the first embodiment and the modification examples thereof. However, in the second embodiment, the detecting unit 17d further performs the following three detecting processes, in addition to the ES time phase detecting process.

In the first embodiment, by using the second selecting method, the detecting unit 17d performs the ES time phase automatic detecting process on the basis of the time-series data of the contour positions obtained as a result of the 2DT process. However, due to a tracking error during the 2DT process, the time phase detecting process performed by the detecting unit 17d may contain an error in some situations. To cope with these situations, as shown in FIG. 12, the detecting unit 17d according to the second embodiment further detects a time phase difference (a difference in ES time phases), which is the difference between end-systolic phases each of which was detected from a different one of the pieces of a plurality of time-series data of contour positions.

Furthermore, the controlling unit 18 performs at least one of the following: a display controlling process to cause the time phase difference to be displayed; and a notification controlling process to cause a notification to be issued if the time phase difference exceeds a predetermined value. For example, the controlling unit 18 causes the monitor 2 to display the time phase difference detected by the detecting unit 17d. Furthermore, if the time phase difference exceeds a predetermined upper limit value, the controlling unit 18 causes the speaker of the monitor 2 to output a beep to prompt the operator to perform the tracking process again or to correct the ES time phase. Alternatively, if the time phase difference exceeds the predetermined upper limit value, the controlling unit 18 causes the monitor 2 to display a message that prompts the operator to perform the tracking process again or to correct the ES time phase. For example, the controlling unit 18 performs the notification controlling process if a “value obtained by dividing the difference (the error) between the ES time phase in the A4C views and the ES time phase in the A2C views by the maximum value among the ES time phases in the A4C views and the ES time phases in the A2C views” exceeds a predetermined set value (e.g., 10%).

Furthermore, the detecting unit 17d according to the second embodiment detects a time period difference indicating the difference in the one-heartbeat periods between the plurality of groups of two-dimensional ultrasound image data, regardless of whether the first selecting method is used or the second selecting method is used. For example, as shown in FIG. 12, the detecting unit 17d according to the second embodiment detects the difference between an R-R interval in the moving image data of the A4C views and an R-R interval in the moving image data of the A2C views. After that, in the same manner as in the example of the time phase difference detection, the controlling unit 18 performs at least one of the following: a display controlling process to cause the time period difference to be displayed; and a notification controlling process to cause a notification to be issued if the time period difference exceeds a predetermined value. For example, the controlling unit 18 performs the notification controlling process if a “value obtained by dividing the difference (the error) between the R-R interval in the A4C views and the R-R interval in the A2C views by the maximum value among the R-R intervals in the A4C views and the R-R intervals in the A2C views” exceeds a predetermined set value (e.g., 5%).

Furthermore, by using the pieces of the plurality of time-series data of contour positions, the detecting unit 17d according to the second embodiment detects a long-axis difference which is the difference between the lengths of the long axis between the plurality of groups of two-dimensional ultrasound image data used in the modified method of the disc summation method (the modified-Simpson's method), regardless of whether the first selecting method is used or the second selecting method is used. For example, the detecting unit 17d detects the difference between the length of the long axis in the A4C view in an ED phase and the length of the long axis in the A2C view in an ED phase. Furthermore, in the same manner as in the examples of the time phase difference detection and the time period difference detection, the controlling unit 18 performs at least one of the following: a display controlling process to cause the long-axis difference to be displayed; and a notification controlling process to cause a notification to be issued if the long-axis difference exceeds a predetermined value. For example, the controlling unit 18 performs the notification controlling process if a “value obtained by dividing the difference (the error) between the length of the long axis in the A4C view and the length of the long axis in the A2C view by the maximum value among the lengths of the long axis in the A4C views and the lengths of the long axis in the A2C views” exceeds a predetermined set value (e.g., 10%).

Furthermore, in the second embodiment, to make it possible for the operator to correct the ES time phase detected by the detecting unit 17d, the following process is performed: The input device 3 receives a change to be made to the end-systolic phase from the operator who has referred to the end-systolic phase detected by the detecting unit 17d on the basis of the time-series data of the contour positions. After that, the volume information calculating unit 17c re-calculates volume information, on the basis of an end-systolic phase resulting from the change received by the input device 3.

For example, when having received a data display request for making a correction from the operator who has referred to a message that prompts the operator to correct the ES time phase, the controlling unit 18 causes the monitor 2 to display the two-dimensional ultrasound image data in a plurality of frames in the time phase detected as the ES time phase and in the time phases before and after the detected time phase, on each of the cross-sectional planes. The operator refers to the displayed plurality of frames on each of the cross-sectional planes and inputs a correction instruction by selecting, with the input device 3, one of the frames which the operator judges to be appropriate to represent the ES time phase. In this situation, when having referred to the displayed plurality of frames on each of the cross-sectional planes, if the operator determines that the time phase detected as the ES time phase is appropriate as the ES time phase, the operator inputs an instruction indicating that no correction is to be made.

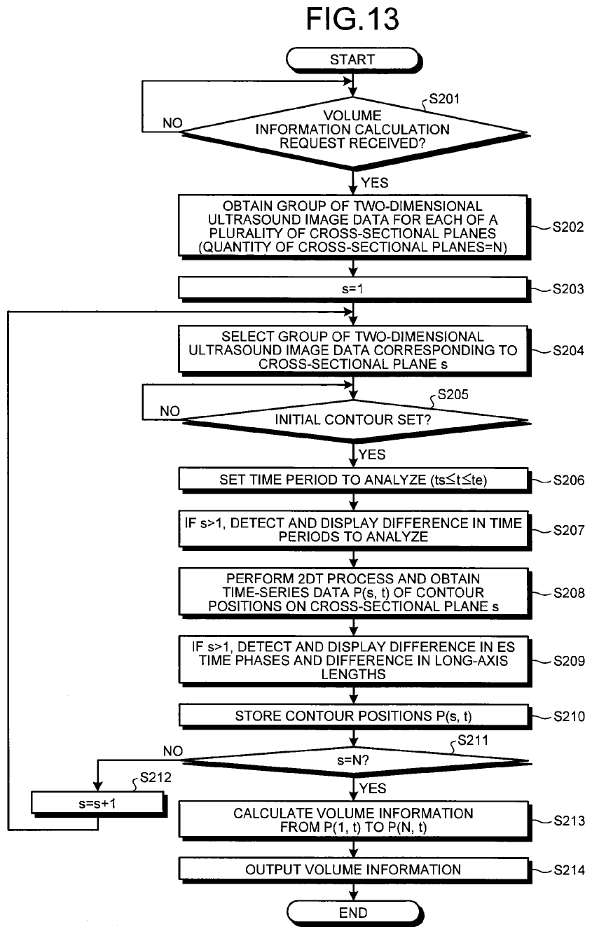

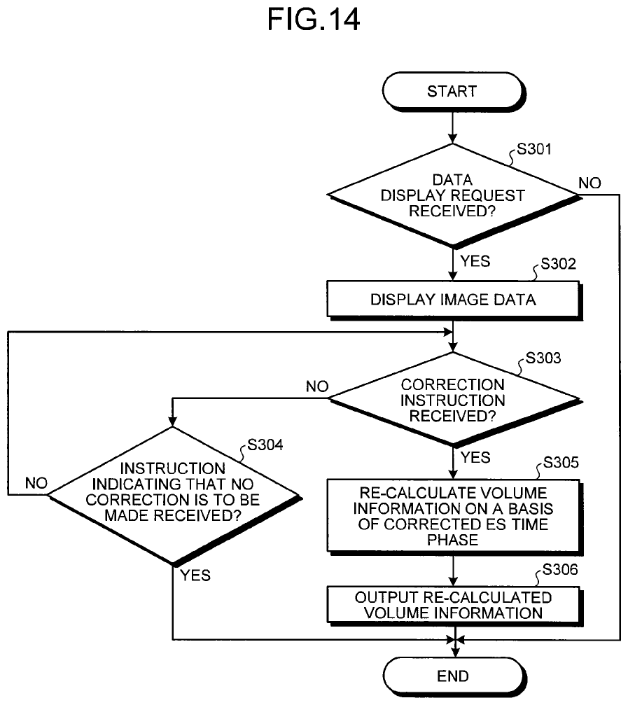

Next, a process performed by the ultrasound diagnosis apparatus according to the second embodiment will be explained, with reference to FIGS. 13 and 14. FIG. 13 is a flowchart for explaining an example of the volume information calculating process performed by the ultrasound diagnosis apparatus according to the second embodiment. FIG. 14 is a flowchart for explaining an example of the volume information re-calculating process performed by the ultrasound diagnosis apparatus according to the second embodiment. FIG. 13 illustrates a flowchart in a situation where an initial contour is set by the operator, and the second selecting method employing the detecting unit 17d is implemented.

As shown in FIG. 13, the ultrasound diagnosis apparatus according to the second embodiment judges whether groups of two-dimensional ultrasound image data each corresponding to a different one of a plurality of cross-sectional planes have been specified as a processing target and whether a volume information calculation request has been received (step S201). In this situation, if a volume information calculation request has not been received (step S201: No), the ultrasound diagnosis apparatus stands by until a volume information calculation request is received.

On the contrary, if a volume information calculation request has been received (step S201: Yes), the image obtaining unit 17a obtains the specified groups of two-dimensional ultrasound image data corresponding to the plurality of cross-sectional planes (where the quantity of cross-sectional planes=N) (step S202). After that, the controlling unit 18 sets “s” so as to satisfy “s=1” (step S203), whereas the contour position obtaining unit 17b selects a group of two-dimensional ultrasound image data corresponding to a cross-sectional plane “s” (step S204). After that, the contour position obtaining unit 17b judges whether an initial contour on the cross-sectional plane “s” has been set (step S205). In this situation, if the initial contour on the cross-sectional plane “s” has not been set (step S205: No), the contour position obtaining unit 17b stands by until the initial contour is set.

On the contrary, if the initial contour has been set (step S205: Yes), the contour position obtaining unit 17b sets a time period to analyze (tste) (step S206). After that, if s>1 is satisfied, the detecting unit 17d detects the difference (the time period difference) in the time periods to analyze, and the monitor 2 displays the difference in the time periods to analyze between the plurality of cross-sectional planes under the control of the controlling unit 18 (step S207). If the difference in the time periods to analyze exceeds a predetermined upper limit value, the monitor 2 displays a message or the like that prompts the operator to perform an analysis by using another piece of moving image data, under the control of the controlling unit 18. When a notification such as a message or the like indicating that the upper limit value is exceeded is output, it is also acceptable for the operator to discontinue the volume information calculating process.

After that, the contour position obtaining unit 17b performs the 2DT process and obtains time-series data P(s,t) of the contour positions on the cross-sectional plane “s” (step S208). Subsequently, the detecting unit 17d detects ES time phases and detects the lengths of the long axis by using P(s,t). After that, if s>1 is satisfied, the detecting unit 17d detects the difference in the ES time phases and the difference in the lengths of the long axis. The monitor 2 displays the difference in the ES time phases and the difference in the lengths of the long axis, under the control of the controlling unit 18 (step S209). If the difference in the ES time phases or the difference in the lengths of the long axis exceeds a predetermined upper limit value, the monitor 2 displays a message or the like that prompts the operator to correct the ES time phase or to perform the analysis again, under the control of the controlling unit 18. When a notification such as a message or the like indicating that the upper limit value is exceeded is output, it is also acceptable for the operator to discontinue the volume information calculating process.

Subsequently, the contour position obtaining unit 17b stores P(s,t) into the internal storage unit 16 (step S210). After that, the contour position obtaining unit 17b judges whether “s=N” is satisfied (step S211). If “s” is not equal to “N” (step S211: No), the contour position obtaining unit 17b sets “s” so as to satisfy “s=s+1” (step S212), and the process returns to step S204 where the contour position obtaining unit 17b selects a group of two-dimensional ultrasound image data on the cross-sectional plane “s”.

On the contrary, if “s=N” is satisfied (step S211: Yes), the volume information calculating unit 17c calculates volume information on the basis of P(1,t) to P(N,t), by using the ES time phases detected by the detecting unit 17d from each of P(1,t) to P(N,t) (step S213). The controlling unit 18 exercises control so that the volume information is output (step S214), and the process ends.

After that, as shown in FIG. 14, the ultrasound diagnosis apparatus according to the second embodiment judges whether a data display request to correct the ES time phase has been received from the operator who has referred to the message that prompts the operator to correct the ES time phase (step S301). In this situation, if a data display request has not been received (step S301: No), the ultrasound diagnosis apparatus according to the second embodiment ends the process.

On the contrary, if a data display request has been received (step S301: Yes), the monitor 2 displays the two-dimensional ultrasound image data in a plurality of frames in the time phase detected as the ES time phase and in the time phases before and after the detected time phase, on each of the cross-sectional planes, under the control of the controlling unit 18 (step S302). Furthermore, the controlling unit 18 judges whether an instruction to correct the ES time phase has been received (step S303). In this situation, if no instruction to correct the ES time phase has been received (step S303: No), the controlling unit 18 judges whether an instruction indicating that no correction is to be made has been received from the operator (step S304). In this situation, if an instruction indicating that no correction is to be made has been received (step S304: Yes), the controlling unit 18 ends the process.

On the contrary, if the instruction indicating that no correction is to be made has not been received (step S304: No), the process returns to step S303 where the controlling unit 18 judges whether an instruction to correct the ES time phase has been received.

If an instruction to correct the ES time phase has been received (step S303: Yes), the volume information calculating unit 17c re-calculates volume information, on the basis of a corrected ES time phase (step S305). After that, the controlling unit 18 outputs the re-calculated volume information (step S306), and the process ends.

As explained above, according to the second embodiment, because there may be an error in the automatic selection of the ES time phase due to an error in the tracking process, the difference between the plurality of cross-sectional planes caused by the automatic detection of the ES time phase is fed back to the operator. In other words, according to the second embodiment, reliability of the tracking result (i.e., the result of the volume information calculating process) is assured by displaying the difference in the ES time phases. In addition, if the difference in the time phases exceeds the predetermined upper limit value, it is possible to, for example, present a message that prompts the operator to correct the ES time phase (or a message that prompts the operator to perform the tracking process again).

Furthermore, in the second embodiment, validity of the image data serving as the analyzed target is assured by displaying the degree of difference in the one-heartbeat periods between the pieces of moving image data. In addition, if the difference in the time periods exceeds the predetermined upper limit value, it is possible to, for example, present a message that prompts the operator to perform an analysis using another piece of moving image data.

Because the notification regarding the difference in the time periods is presented, it is possible to reduce the errors that may occur during the operator's operation to specify a desired piece of data from among a plurality of candidates of moving image data of the same subject that are displayed by a viewing tool, when selecting the moving image data to be used in the analysis. More specifically, a large number of pieces of data having mutually-different heart rates are mixed together among a series of moving image data obtained during a stress echo test, due to a variation in the stress status. As another example, in the medical case of atrial fibrillation, because the R-R period fluctuates significantly, a large number of heartbeat periods that vary from one another are displayed by a viewing tool, in a plurality of pieces of moving image data taken on mutually-different cross-sectional planes. In these situations, by presenting the notification regarding the difference in the time periods as explained in the second embodiment, it is possible to reduce the errors that may occur during the data specifying operation.

Furthermore, as explained above, when the “modified-Simpson's method” is used, the degree of difference in the lengths of the long axis of the left ventricle is important in assuring the reliability of the volume information. For this reason, in the second embodiment, validity of the image data serving as the analyzed target is assured by displaying the degree of difference in the lengths of the long axis between the pieces of moving image data. In addition, if the long-axis difference exceeds the predetermined upper limit value, it is possible to, for example, present a message that prompts the operator to perform the analysis again or to perform an analysis using another piece of moving image data.

As explained above, according to the second embodiment, by detecting and outputting the various types of difference information that may be a cause of a decrease in the level of precision in the volume information calculating process, it is possible to further improve the precision level of the volume information calculating process.

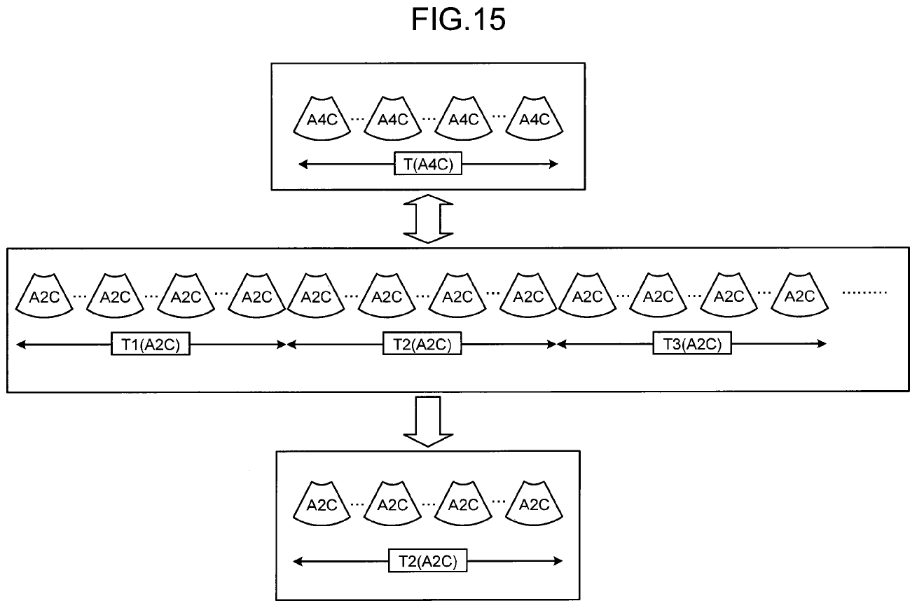

The second embodiment may be realized in the following modification example, for the purpose of avoiding the cause of a decrease in the precision level in the volume information calculating process. FIG. 15 is a drawing for explaining a modification example of the second embodiment.

The image obtaining unit 17a according to the present modification example obtains groups of two-dimensional ultrasound image data having substantially equal one-heartbeat periods, by obtaining one group from each of a plurality of groups of two-dimensional ultrasound image data. For example, as shown in FIG. 15, let us assume that the R-R interval of the moving image data of A4C views for a one-heartbeat period on which the 2DT process has been performed was “T(A4C)”. Also, for example, as shown in FIG. 15, let us assume that the moving image data of the A4C views is moving image data for a three-heartbeat period. In that situation, as shown in FIG. 15, the image obtaining unit 17a calculates three R-R intervals “T1(A2C), T2(A2C), T3(A2C)” each corresponding to a one-heartbeat period, from the moving image data of A2C views for the three-heartbeat period. Furthermore, as shown in FIG. 15 for example, the image obtaining unit 17a outputs, to the contour position obtaining unit 17b, moving image data of A2C views for a one-heartbeat period corresponding to “T2(A2C)”, which has the smallest difference from “T(A4C)”.

In the present modification example, it is acceptable, for example, to configure the image obtaining unit 17a so as to obtain pieces of moving image data for a one-heartbeat period having substantially equal R-R intervals, from moving image data of A4C views for a multiple-heartbeat period and from moving image data of A2C views for a multiple-heartbeat period and so as to output the obtained pieces of moving image data to the contour position obtaining unit 17b. Alternatively, it is acceptable, for example, to configure the image obtaining unit 17a so as to obtain pieces of moving image data for a three-heartbeat period having substantially equal R-R intervals, from moving image data of A4C views for a multiple-heartbeat period and from moving image data of A2C views for a multiple-heartbeat period and so as to output the obtained pieces of moving image data to the contour position obtaining unit 17b. In those situations, as explained in the first modification example of the first embodiment, the volume information calculating unit 17c calculates average volume information, from the time-series data of the contour positions for the three-heartbeat period in the A4C views and from the time-series data of the contour positions for the three-heartbeat period in the A2C views. Alternatively, it is acceptable, for example, to configure the image obtaining unit 17a so as to obtain a plurality of pairs of moving image data for a one-heartbeat period having substantially equal R-R intervals, from moving image data of A4C views for a multiple-heartbeat period and from moving image data of A2C views for a multiple-heartbeat period and so as to output the obtained pairs of moving image data to the contour position obtaining unit 17b. In that situation, the volume information calculating unit 17c calculates volume information for each of the pairs.

By using the present modification example, it is possible to automate the process to select the moving image data serving as the analyzed target. It is therefore possible to further reduce the burden on the operator required during the volume information calculating process. The processes explained in the second embodiment and the modification examples described above is applicable to a situation where the volume information is calculated by using the “biplane area-length method”, instead of the “modified-Simpson's method”.

As a third embodiment, an example in which a temporal change curve of the volume is calculated as volume information will be explained.

The image processing unit 17 according to the third embodiment has the same configuration as that of the image processing unit 17 according to the first embodiment shown in FIG. 4. In other words, the image processing unit 17 according to the third embodiment includes the image obtaining unit 17a, the contour position obtaining unit 17b, the volume information calculating unit 17c, and the detecting unit 17d that are configured to perform the processes explained in the first embodiment, the modification examples thereof, the second embodiment, and the modification examples thereof. However, in the third embodiment, in addition to EDV, ESV, EF, the myocardial mass, and the like, the volume information calculating unit 17c further calculates time-series data of volume information (a temporal change curve of the volume information) on the basis of pieces of a plurality of time-series data of contour positions. In this situation, the volume information calculating unit 17c calculates the time-series data of the volume information by using either the “modified-Simpson's method” or the “biplane area-length method”. After that, the controlling unit 18 causes the temporal change curve of the volume information to be output.

For example, the volume information calculating unit 17c calculates a temporal change curve of the volume of the left ventricular cavity interior, from pieces of a plurality of time-series data of contour positions. Alternatively, for example, the volume information calculating unit 17c calculates a temporal change curve of the myocardial mass from pieces of a plurality of time-series data of contour positions. In this situation, if incompressibility of the myocardium is assumed, the temporal change in values of the myocardial mass within a cardiac cycle is small. Thus, it is appropriate to use a value in an end-diastolic phase as a representative value. However, in the third embodiment, because the time-series data of the volume information is calculated and output, it is also a good idea to output the temporal change curve of the myocardial mass for the purpose of analyzing the myocardial mass in detail.

When calculating the temporal change curve of the volume information described above, however, the volume information calculating unit 17c needs to calculate a value of the volume for a period of at least one heartbeat in each of all the cardiac phases. In this situation, when pieces of moving image data on a plurality of cross-sectional planes are acquired at the same time by performing a simultaneous scan on a plurality of cross-sectional planes (e.g., an A4C view and an A2C view) by using a 2D array probe, the volume information calculating unit 17c is able to calculate values of the volume in mutually the same cardiac phase, on the basis of the pieces of moving image data. However, when a plurality of pieces of moving image data acquired in mutually-different time phases by using a 1D array probe are used, there is a possibility that the pieces of moving image data may not contain pieces of image data that are in mutually the same cardiac phase. In other words, one-heartbeat periods may be different among the plurality of pieces of moving image data due to variations in the heartbeats. In addition, between a plurality of pieces of moving image data taken on mutually-different cross-sectional planes, the frame rate setting may be different among the plurality pieces of moving image data, due to variations in conditions such as the scanning angle, or the like. To cope with these situations, in the third embodiment, when calculating a value of the volume on the basis of contour information in a certain cardiac phase, it is necessary to, in consideration of these temporal variation factors, calculate the volume after temporally interpolating the contour position in a piece of image data having the same time phase as another piece of image data, among the group of moving image data.

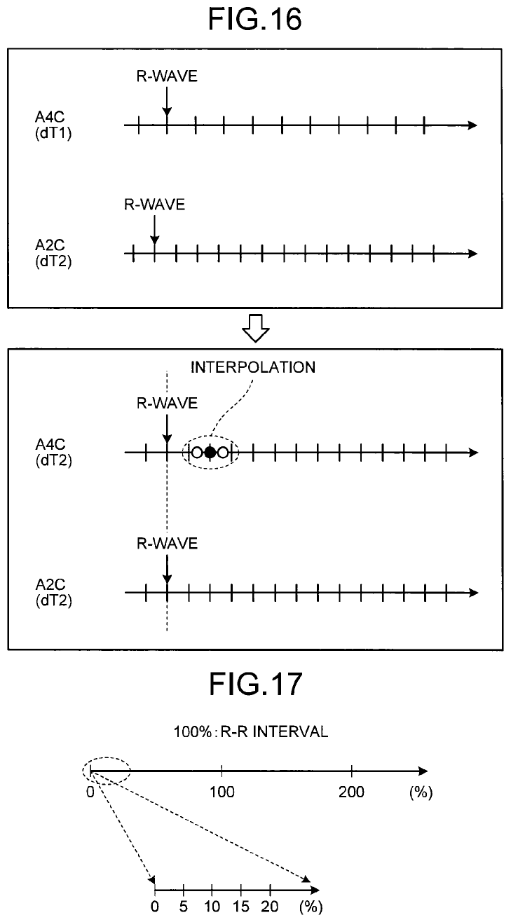

Thus, in the third embodiment, when calculating temporal change information of the volume information, the contour position obtaining unit 17b performs a temporal interpolation process to correct each of the pieces of the plurality of time-series data of contour positions, so as to obtain synchronized pieces of time-series data that have contour positions in substantially mutually-the-same time phase. Examples of interpolating methods include the following two methods. FIGS. 16 and 17 are drawings for explaining the contour position obtaining unit according to the third embodiment.

First, a first interpolating method will be explained with reference to FIG. 16. In the example illustrated in FIG. 16, it is assumed that the frame interval of the moving image data of A4C views is “dT1”, whereas the frame interval of the moving image data of A2C views is “dT2” (where dT2<dT1) (see the upper chart in FIG. 16).

According to the first interpolating method, as shown in the lower chart in FIG. 16, for example, the contour position obtaining unit 17b aligns the starting points of the time-series data of the contour positions in the A4C views and the time-series data of the contour positions in the A2C views, with an R-wave time phase, which is used as a reference phase. Alternatively, a P-wave phase, which corresponds to the beginning of a contraction of an atrium, may be used as the reference phase.

For example, the contour position obtaining unit 17b determines the time-series data of the contour positions in the A4C view, which has a longer frame interval, to be a target of the interpolation process. After that, the contour position obtaining unit 17b calculates, by performing an interpolation process, a contour position in an A4C view in the same time phase (the same elapsed time period since the R-wave time phase) as the contour position in the A2C views obtained at the “dT2” interval, by using contour positions in A4C views obtained near the same time phase (the elapsed time period) (see the oval with a broken line in the lower chart in FIG. 16). In the example shown in the lower chart in FIG. 16, by performing the interpolation process, the contour position obtaining unit 17b calculates a contour position in the time phase indicated with one block dot on the basis of the two contour positions obtained in the time phases indicated with two white dots. As a result, the contour position obtaining unit 17b generates time-series data of the contour positions in the A4C views having a temporal resolution “dT2”, which is the same as that of the time-series data of the contour positions in the A2C views. Consequently, the contour position obtaining unit 17b is able to arrange the time-series data of the contour positions in the A4C views and the time-series data of the contour positions in the A2C views to be the synchronized pieces of time-series data.

In contrast, according to a second interpolating method, the contour position obtaining unit 17b relatively matches the intervals in a reference time phase between the time-series data of the contour positions in the A4C views and the time-series data of the contour positions in the A2C views. For example, according to the second interpolating method, as shown in FIG. 17, the time-series data of the contour positions in the A4C views is arranged to be time-series data assuming the R-R interval of the subject P during the acquisition of the A4C views to be 100%. Furthermore, according to the second interpolating method, as shown in FIG. 17, the time-series data of the contour positions in the A2C views is arranged to be time-series data assuming the R-R interval of the subject P during the acquisition of the A2C views to be 100%. Furthermore, the contour position obtaining unit 17b sets a plurality of relative elapsed time periods (e.g., 5%, 10%, 15%, 20%, and so on) obtained by dividing the time period in the reference time phase assumed to be 100% into sections of a predetermined length.

After that, with regard to the time-series data of the contour positions in the A4C views, the contour position obtaining unit 17b calculates, by performing an interpolation process, a contour position in each of the relative elapsed time periods, while using the contour position in an A4C view obtained near each of the relative elapsed time periods. With regard to the time-series data of the contour positions in the A2C views, the contour position obtaining unit 17b calculates, by performing an interpolation process, a contour position in each of the relative elapsed time periods, while using the contour position in an A2C view obtained near each of the relative elapsed time periods.

Subsequently, to convert the relative elapsed time periods (%) into absolute time periods (milliseconds), the contour position obtaining unit 17b multiplies the relative elapsed time periods (%) by “the R-R interval during the acquisition of the A4C views/100” or “the R-R interval during the acquisition of the A2C views/100”. Alternatively, the contour position obtaining unit 17b may multiply the relative elapsed time periods (%) by “(an average of the R-R interval during the acquisition of the A4C view and the R-R interval during the acquisition of the A2C views)/100”. As a result, the contour position obtaining unit 17b is able to arrange the time-series data of the contour positions in the A4C views and the time-series data of the contour positions in the A2C views to be the synchronized pieces of time-series data.

Consequently, the volume information calculating unit 17c is able to calculate, for example, volumes of the cavity interior in mutually the same time phase or myocardial masses in mutually the same time phase.

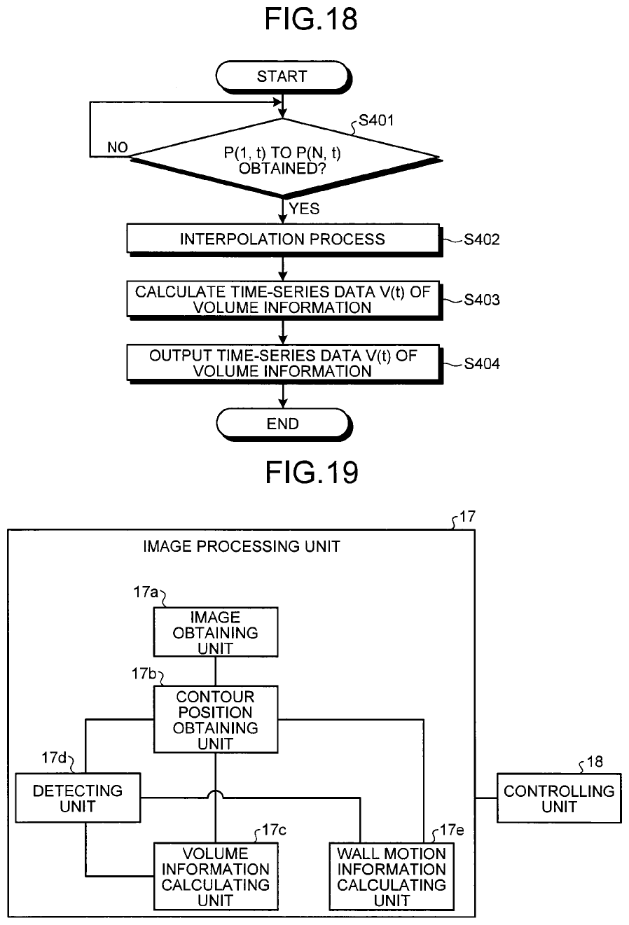

Next, a process performed by the ultrasound diagnosis apparatus according to the third embodiment will be explained, with reference to FIG. 18. FIG. 18 is a flowchart for explaining an example of the process performed by the ultrasound diagnosis apparatus according to the third embodiment. FIG. 18 illustrates a process that is triggered when pieces of time-series data of contour positions on all of the plurality of cross-sectional planes have been obtained as a result of the process explained in the first embodiment or the second embodiment.

As shown in FIG. 18, the ultrasound diagnosis apparatus according to the third embodiment judges whether P(1,t) to P(N,t) have been obtained (step S401). In this situation, if P(1,t) to P(N,t) have not all been obtained (step S401: No), the ultrasound diagnosis apparatus stands by until the time-series data of the contour positions on all of the plurality of cross-sectional planes have been obtained.

On the contrary, if P(1,t) to P(N,t) have all been obtained (step S401: Yes), the contour position obtaining unit 17b performs an interpolation process by using either the first interpolating method or the second interpolating method (step S402). After that, the volume information calculating unit 17c calculates time-series data V(t) of volume information on the basis of P(1,t) to P(N,t), by using an ES time phase of each of P(1,t) to P(N,t) detected by the detecting unit 17d (step S403). After that, the controlling unit 18 exercises control so that the time-series data V(t) of the volume information is output (step S404), and the process ends.

As explained above, according to the third embodiment, it is possible to calculate the time-series data of the volume information with an excellent level of precision, by performing the interpolation process on the contour positions.

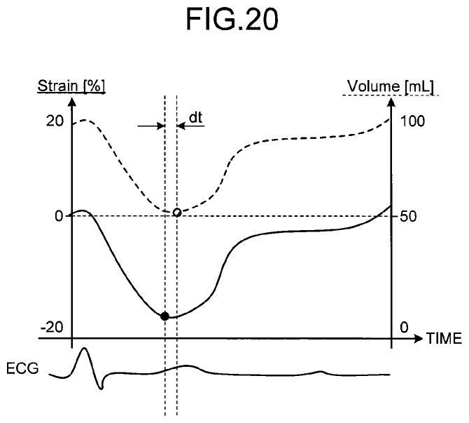

As a fourth embodiment, with reference to FIGS. 19 and 20, an example will be explained in which wall motion information is further calculated by using the time-series data of the contour positions on the plurality of cross-sectional planes. FIG. 19 is a block diagram of an exemplary configuration of an image processing unit according to the fourth embodiment. FIG. 20 is a drawing of an example of information that is output according to the fourth embodiment.

As shown in FIG. 19, the image processing unit 17 according to the fourth embodiment further includes a wall motion information calculating unit 17e, being different from the image processing unit 17 according to the first embodiment shown in FIG. 4. In other words, the image processing unit 17 according to the fourth embodiment includes the image obtaining unit 17a, the contour position obtaining unit 17b, the volume information calculating unit 17c, and the detecting unit 17d that are configured to perform the processes explained in the first to the third embodiments and the modification examples thereof, and also includes the wall motion information calculating unit 17e.

Generally speaking, during the 2DT process, information about a strain in a myocardium and the like is obtained as wall motion information. It is desirable if such wall motion information is output as a temporal change curve. In the fourth embodiment, by utilizing the configuration described in the first to the third embodiments where it is possible to track the contour positions by performing the 2DT process, the wall motion information is obtained at the same time and is output at the same time as the volume information.

More specifically, the wall motion information calculating unit 17e shown in FIG. 19 calculates wall motion information of a predetermined site, on the basis of pieces of a plurality of time-series data of contour positions. After that, the controlling unit 18 exercises control so that the volume information and the wall motion information are output.

More specifically, the wall motion information calculating unit 17e calculates at least one of the following as the wall motion information: a local strain; a local displacement; a rate of temporal changes in a local strain (a “strain rate”); a rate of temporal changes in a local displacement (a “velocity”); an overall strain; an overall displacement; a rate of temporal changes in an overall strain; and a rate of temporal changes in an overall displacement. For example, the wall motion information calculating unit 17e calculates wall motion information in an ES time phase, on the basis of a contour position in the ES time phase detected by the detecting unit 17d explained in the first embodiment. Alternatively, the wall motion information calculating unit 17e may calculate time-series data of the wall motion information. When the wall motion information calculating unit 17e calculates the time-series data of the wall motion information, the contour position obtaining unit 17b corrects the pieces of time-series data of the contour positions each of which corresponds to a different one of the plurality of cross-sectional planes, so as to obtain synchronized pieces of time-series data by performing the interpolation process explained in the third embodiment.

For example, on the basis of a result of a 2DT process performed on the inner layer and the outer layer on an A4C cross-sectional plane and an A2C cross-sectional plane, the wall motion information calculating unit 17e calculates, as the wall motion information, a local strain in a longitudinal direction (LS), a local strain in a circumferential direction (CS), and a local strain in a wall-thickness (radial) direction (RS). Alternatively, for example, on the basis of a result of a 2DT process performed on the inner layer and the outer layer on an A4C cross-sectional plane and an A2C cross-sectional plane, the wall motion information calculating unit 17e calculates, as the wall motion information, an overall strain by averaging the local strains on the A4C cross-sectional plane and the A2C cross-sectional plane described above. Furthermore, the wall motion information calculating unit 17e calculates a rate of temporal changes in the local strain and a rate of temporal changes in the overall strain.

For example, on the basis of a result of the 2DT process performed on the inner layer and the outer layer on an A4C cross-sectional plane or an A2C cross-sectional plane, the wall motion information calculating unit 17e calculates, as the wall motion information, a regional longitudinal displacement (LD) and a regional radial (wall-thickness direction) displacement (RD). Alternatively, for example, on the basis of a result of the 2DT process performed on the inner layer and the outer layer on an A4C cross-sectional plane and an A2C cross-sectional plane, the wall motion information calculating unit 17e calculates, as the wall motion information, an overall displacement by averaging the local displacements on the A4C cross-sectional plane and the A2C cross-sectional plane described above. Furthermore, the wall motion information calculating unit 17e calculates a rate of temporal changes in the local displacement (a local myocardial velocity) and a rate of temporal changes in the overall displacement (an overall myocardial velocity). When the displacements are used as the wall motion information, the wall motion information calculating unit 17e may calculate a moving distance of a tracking point (an absolute displacement (AD)) in a time phase other than a reference time phase (e.g., an R-wave), with respect to the position of the tracking point in the reference time phase.

One or more types of wall motion information to be calculated by the wall motion information calculating unit 17e are specified by the operator. Alternatively, one or more types of wall motion information to be calculated by the wall motion information calculating unit 17e may be initially set according to a state stored in a system.

In this situation, under the control of the controlling unit 18, as shown in FIG. 20 for example, the volume information calculating unit 17c generates a temporal change curve of the volume (volume (mL)) of the cavity interior. Furthermore, as shown in FIG. 20 for example, the wall motion information calculating unit 17e generates a temporal change curve of the LS (%). Furthermore, under the control of the controlling unit 18, as shown in FIG. 20 for example, the volume information calculating unit 17c, the wall motion information calculating unit 17e, or the image generating unit 14 generates a chart in which the temporal change curve of the volume of the cavity interior and the temporal change curve of the LS are superimposed together.

After that, the controlling unit 18 causes the monitor 2 to display a chart illustrated in FIG. 20, for example. The result of the volume measuring process using the plurality of cross-sectional planes indicated in the chart in FIG. 20 is mainly used for assuring the precision level of an estimated volume in a medical case exhibiting a local wall motion abnormality that often involves a local shape deformation. Furthermore, the result of the myocardial strain measuring process indicated in the chart in FIG. 20 is used as an index for evaluating the degree of wall motion abnormalities with ischemic heart diseases or diseases involving asynchrony. As a result of the concurrent display of the volume information and the strain information in the chart shown in FIG. 20, the operator is able to make a more detailed diagnosis of cardiac functions easily and accurately than in the situation where only the volume information is output.

As another example, according to the fourth embodiment, under the control of the controlling unit 18, either the volume information calculating unit 17c or the wall motion information calculating unit 17e may, as shown in FIG. 20, calculate a time difference (see “dt” in FIG. 20) between a peak of the volume (the minimum volume) and a peak of the strain (the minimum of the LS), from the chart showing the two temporal change curves obtained in mutually the same cardiac phase. In that situation, the controlling unit 18 also outputs the time difference “dt” between the two peak times, together with the chart. It is possible to calculate the temporal change curves of the volume and the wall motion information and the time difference between the peak times shown in FIG. 20, at each of different times such as before a treatment, after the treatment, and when regular medical examinations are performed after the treatment. By making a comparison of these results during a treatment process, the operator is able to make use of the information for judging the effectiveness of the treatment.

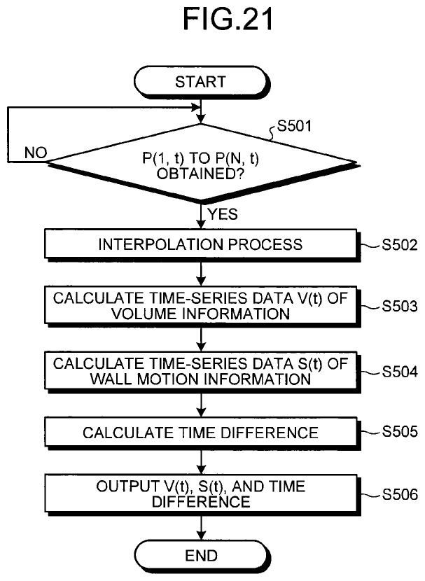

Next, a process performed by the ultrasound diagnosis apparatus according to the fourth embodiment will be explained, with reference to FIG. 21. FIG. 21 is a flowchart for explaining an example of the process performed by the ultrasound diagnosis apparatus according to the fourth embodiment. FIG. 21 illustrates a process that is triggered when pieces of time-series data of contour positions on all of the plurality of cross-sectional planes have been obtained as a result of the process explained in the first embodiment or the second embodiment. Furthermore, in FIG. 21, an example in which time-series data is calculated as the wall motion information is explained.

As shown in FIG. 21, the ultrasound diagnosis apparatus according to the fourth embodiment judges whether P(1,t) to P(N,t) have been obtained (step S501). In this situation, if P(1,t) to P(N,t) have not all been obtained (step S501: No), the ultrasound diagnosis apparatus stands by until the time-series data of the contour positions on all of the plurality of cross-sectional planes have been obtained.

On the contrary, if P(1,t) to P(N,t) have all been obtained (step S501: Yes), the contour position obtaining unit 17b performs an interpolation process by using either the first interpolating method or the second interpolating method (step S502). After that, the volume information calculating unit 17c calculates time-series data V(t) of volume information on the basis of P(1,t) to P(N,t), by using an ES time phase of each of P(1,t) to P(N,t) detected by the detecting unit 17d (step S503).

Furthermore, the wall motion information calculating unit 17e calculates time series data S(t) of wall motion information on the basis of P(1,t) to P(N,t), by using the ES time phase of each of P(1,t) to P(N,t) detected by the detecting unit 17d (step S504). Subsequently, the wall motion information calculating unit 17e calculates a time difference between a peak time of the volume and a peak time of the wall motion information (step S505).

After that, the controlling unit 18 exercises control so that V(t), S(t), and the time difference are output (step S506), and the process ends.

As explained above, according to the fourth embodiment, the wall motion information and the information (the time difference) that can be detected from the volume information and the wall motion information are output, together with the volume information. Thus, the operator is able to easily obtain the various types of information that are important and have a high level of precision to be used in a diagnosis process of heart diseases.

The image processing methods explained in the first to the fourth embodiments and the modification examples thereof are applicable to situations where an organ (e.g., the liver) other than the heart or a tumor occurring in an organ is used as the target of which the volume information is calculated. In those situations, it is possible to automatically track the position of the tumor by performing the 2DT process, even if the tumor moves in the images due to pulsation or respiration. As a result, it is possible to accurately evaluate the state of changes in the volume with respect to the entire tumor or a specific site in the tumor, during a one-heartbeat period or a multiple-heartbeat period, without being influenced by the moving of the position.

The image processing methods explained in the first to the fourth embodiments and the modification examples thereof may be implemented by using a plurality of groups of two-dimensional medical image data each of which is taken on a different one of a plurality of predetermined cross-sectional planes, in a time period equal to or longer than one heartbeat, while employing a medical image diagnosis apparatus (e.g., an X-ray Computer Tomography (CT) apparatus, a Magnetic Resonance Imaging (MRI) apparatus) other than the ultrasound diagnosis apparatus. In other words, because it is possible to perform the 2DT process using the pattern matching process on two-dimensional X-ray CT image data or two-dimensional MRI image data, the image processing methods explained in the first to the fourth embodiments and the modification examples thereof may be implemented by a medical image diagnosis apparatus other than the ultrasound diagnosis apparatus.

Furthermore, the image processing methods explained in the first to the fourth embodiments and the modification examples thereof may be implemented by an image processing apparatus that is provided independently of a medical image diagnosis apparatus. In that situation, the image processing apparatus implements any of the image processing methods described above after receiving a plurality of groups of two-dimensional medical image data received from the medical image diagnosis apparatus, from a database of a Picture Archiving and Communication System (PACS), or from a database of an electronic medical record system.

Furthermore, it is possible to realize any of the image processing methods explained in the first to the fourth embodiments and the modification examples thereof, by causing a computer such as a personal computer or a workstation to execute an image processing computer program (hereinafter, the “image processing program”) prepared in advance. It is possible to distribute the image processing program via a network such as the Internet. Furthermore, it is also possible to record the image processing program onto a computer-readable non-transitory recording medium such as a hard disk, a flexible disk (FD), a Compact Disk Read-Only Memory (CD-ROM), a Magneto-optical (MO) disk, a Digital Versatile Disk (DVD), or a flash memory such as a Universal Serial Bus (USB) memory or a Secure Digital (SD) card memory, so that a computer reads the image processing program from the non-transitory recording medium and executes the read program.

As explained above, according to an aspect of the first to the fourth embodiments and the modification examples thereof, it is possible to easily obtain the measured results of the volume information having a high level of precision.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasound diagnosis apparatus, comprising:
processing circuitry configured to
obtain a first group of two-dimensional ultrasound image data corresponding to a first cross-sectional plane and a second group of two-dimensional ultrasound image data corresponding to a second cross-sectional plane intersecting the first cross-sectional plane;
obtain first time-series data of a contour position from the first group of two-dimensional ultrasound image data and second time-series data of the contour position from the second group of two-dimensional ultrasound image data, the contour position being either one of, or both of, a cavity interior and a cavity exterior in a heart;
identify a first contour position of an end-diastole phase and a second contour position of an end-systole phase based on the contour position of the first time-series data, a third contour position of an end-diastole phase and a fourth contour position of an end-systole phase based on the contour position of the second time-series data; and
calculate an ejection fraction of a predetermined site based on the first contour position and the second contour position and the third contour position and the fourth contour position.

2. The ultrasound diagnosis apparatus according to claim 1,
wherein the processing circuitry is further configured to detect, from each of the first time-series data of contour position and the second time-series data of the contour position, the end-systolic phase;
detect a time phase difference, with the time phase difference being a difference in end-systolic phases detected from each of the first time-series data of the contour position and the second time-series data of the contour position; and
perform a notification controlling process to cause a notification which prompts an operator to perform at least one of obtaining the first time-series data of the contour position and the second time-series data of the contour position and correcting the end-systolic phase to be issued when the time phase difference exceeds a predetermined value.

3. The ultrasound diagnosis apparatus according to claim 1, wherein the processing circuitry is further configured to
obtain the first time-series data of the contour position and the second time-series data of the contour position, with at least one of ventricles and atria of the heart.

4. The ultrasound diagnosis apparatus according to claim 3, further comprising input hardware configured to receive a setting of an end-systolic phase, wherein
the processing circuitry is further configured to select, based on information about the setting received by the input hardware, and based on each of the first time-series data of the contour position and the second time-series data of the contour position, a contour position in an end-systolic phase, and calculate, by using the selected contour position, the representative value of strains at the end-systolic phase.

5. The ultrasound diagnosis apparatus according to claim 3, wherein the processing circuitry is further configured to
select, from each of the first time-series data of the contour position and the second time-series data of the contour position, based on the end systolic phase, a contour position in an end-systolic phase; and
calculate, by using the selected contour position, the representative value of strains at the end-systolic phase.

6. The ultrasound diagnosis apparatus according to claim 5, wherein the processing circuitry is further configured to
perform a display controlling process to cause the time phase difference to be displayed.

7. The ultrasound diagnosis apparatus according to claim 1, wherein the processing circuitry is further configured to
detect a time period difference that is a time difference in periods that are defined as lasting one heartbeat, in the first group of two-dimensional ultrasound image data and the second group of two-dimensional ultrasound image data, and
perform at least one of a display controlling process to cause the time period difference to be displayed and a notification controlling process to cause a notification to be issued when the time period difference exceeds a predetermined value.

8. The ultrasound diagnosis apparatus according to claim 1, wherein the processing circuitry is further configured to
perform a temporal interpolation process to correct each of the first time-series data of the contour position and the second time-series data of the contour position so as to obtain synchronized pieces of time-series data that have contour positions in a same time phase, and
calculate the ejection fraction of a predetermined site based on the corrected first time-series data of the contour position and the corrected second time-series data of the contour position.

9. The ultrasound diagnosis apparatus according to claim 1, wherein the processing circuitry is further configured to extract groups of two-dimensional ultrasound image data having equal one-heartbeat periods, by obtaining one group from each of the first group of two-dimensional ultrasound image data and the second group of two-dimensional ultrasound image data.

10. An image processing apparatus, comprising:
processing circuitry configured to
obtain a first group of two-dimensional medical image data and a second group of two-dimensional ultrasound image data, the first group being taken on a first cross-sectional plane and the second group being taken on a second cross-sectional plane;
obtain first time-series data of a contour position from the first group of two-dimensional ultrasound image data and second time-series data of the contour position from the second group of two-dimensional ultrasound image data, the contour position being either one of, or both of, a cavity interior and a cavity exterior in a heart;
identify a first contour position of an end-diastole phase and a second contour position of an end-systole phase based on the contour position of the first time-series data, a third contour position of an end-diastole phase and a fourth contour position of an end-systole phase based on the contour position of the second time-series data; and
calculate an ejection fraction of a predetermined site based on the first contour position and the second contour position and the third contour position and the fourth contour position.

11. A image processing method executed by a computer, the method comprising:
obtaining a first group of two-dimensional ultrasound image data corresponding to a first cross-sectional plane and a second group of two-dimensional ultrasound image data corresponding to a second cross-sectional plane intersecting the first cross-sectional plane;
obtaining first time-series data of a contour position from the first group of two-dimensional ultrasound image data and second time-series data of the contour position from the second group of two-dimensional ultrasound image data, the contour position being either one of, or both of, a cavity interior and a cavity exterior in a heart;
identifying a first contour position of an end-diastole phase and a second contour position of an end-systole phase based on the contour position of the first time-series data, a third contour position of an end-diastole phase and a fourth contour position of an end-systole phase based on the contour position of the second time-series data; and
calculating an ejection fraction of a predetermined site based on the first contour position and the second contour position and the third contour position and the fourth contour position.

12. The ultrasound diagnosis apparatus according to claim 1, wherein a tracking process performed by the processing circuitry includes a two-dimensional pattern matching process.

13. The ultrasound diagnosis apparatus according to claim 1, wherein the processing circuitry is further configured to align a starting point of the first time-series data of the contour position with a starting point of the second time-series data of the contour position in a cardiac phase.

14. The ultrasound diagnosis apparatus according to claim 1, wherein the first cross-sectional plane corresponds to an apical four-chamber view and the second cross-sectional plane corresponds to an apical two-chamber view.

15. The ultrasound diagnosis apparatus according to claim 1, wherein the processing circuitry is further configured to
obtain, by performing a tracking process, the first time-series data of the contour position from the first group of two-dimensional ultrasound image data and the second time-series data of the contour position from the second group of two-dimensional ultrasound image data.
